(19) United States
(12) Patent Application Publication
(10) Pub. No.: US 2016/0096931 A1
(43) Pub. Date: Apr. 7, 2016

(54) COMPOSITE MATERIALS CONTAINING STRUCTURAL POLYSACCHARIDES AND MACROCYCLIC COMPOUNDS FORMED FROM IONIC LIQUID COMPOSITIONS

(71) Applicant: MARQUETTE UNIVERSITY, Milwaukee, WI (US)

(72) Inventor: Chieu D. Tran, Fox Point, WI (US)

(73) Assignee: Marquette University, Milwaukee, WI (US)

(21) Appl. No.: 14/891,896

(22) PCT Filed: May 16, 2014
(86) PCT No.: PCT/US14/38381

§ 371 Date: Nov. 17, 2015

Related U.S. Application Data
(60) Provisional application No. 61/824,717, filed on May 17, 2013.

(51) Int. Cl. C08J 3/11; C08J 3/09; B01J 20/30; B01D 15/26; A61L 15/28; B01D 39/16; C02F 1/28; A01N 43/04; B01J 20/24

(52) U.S. Cl. CPC C08J 3/11; A01N 43/04; C08J 3/096; B01J 20/30; B01J 20/24; A61L 15/28; B01D 39/16; C02F 1/286; B01D 15/26; C08J 2301/02; C08J 2305/08; C08J 2305/16; C02F 2101/363

(57) ABSTRACT

Disclosed herein are composite materials, ionic liquid compositions for preparing the composite materials, and methods for using the composite materials prepared from the ionic liquid compositions. The composite materials typically include structural polysaccharides and preferably include macrocyclic compounds. The composite materials may be prepared from ionic liquid compositions comprising the structural polysaccharides and preferably the macrocyclic compounds dissolved in the ionic liquid, where the ionic liquid is removed from the ionic liquid compositions to obtain the composite materials.

### CROSS-REFERENCE TO RELATED PATENT APPLICATIONS

The present application claims the benefit of priority under 35 U.S.C. §119(e) to U.S. Provisional Patent Application No. 61/824,717, the content of Which is incorporated herein by reference in its entirety.

### STATEMENT REGARDING FEDERALLY SPONSORED RESEARCH OR DEVELOPMENT

This invention was made with government support under R15GM-99033 awarded by the National Institutes of Health. The government has certain rights in the invention.

### BACKGROUND

The field of the invention relates to composite materials containing structural polysaccharides and macrocylic compounds and ionic liquid composition for preparing the composite materials. In particular, the field of the invention relates to composite materials containing structural polysaccharides, such as cellulose, chitin, or chitosan, and macrocylic compounds, such as cyclodextrins, formed from ionic liquid compositions.

### SUMMARY

Disclosed herein are composite materials comprising one or more structural polysaccharides and preferably one or more macrocyclic compounds. The composite materials may be prepared from ionic liquid compositions comprising the one or more polysaccharides dissolved in the one or more ionic liquids and preferably the one or more macrocyclic compounds dissolved in the one or more ionic liquids. The composite materials may be prepared from the ionic liquid compositions, for example, by removing the ionic liquid from the ionic liquid composition and retaining the one or more structural polysaccharides and preferably the one or more macrocyclic compounds.

The disclosed compositions typically comprise one or more structural poly which may include, but are not limited to polymers such as polysaccharides comprising monosaccharides linked via beta-1,4 linkages. For example, structural polysaccharides may include polymers of 6-carbon monosaccharides linked via beta-1,4 linkages. Suitable structural polysaccharides for the disclosed compositions may include, but are not limited to cellulose, chitin, and modified forms of chitin such as chitosan.

The disclosed compositions preferably comprise one or more macrocyclic compounds. Suitable macrocyclic compounds may include but are not limited to cyclodextrins, calixarenes, carcerands, crown ethesr, cyclophanes, cryptands, cucurbiturils, pillararenes, and spherands.

In some embodiments, the macrocyclic compound is a cyclodextrin. In further embodiments cyclodextrin is an α-cyclodextrin, a β-cyclodextrin, or a γ-cyclodextrin. The cyclodextrin may be modified for example, by haying one or more substitutions on a hydroxyl group, such as, a substitution on one or more of the 2-hydroxyl group, the 3-hydroxyl group, and the 6-hydroxyl group of any glucose monomer of the cyclodextrin. Suitable substitutions may include, but are not limited to alkyl group substitutions (e.g., methyl substitutions), a hydroxyalkyl group substitution, a sulfoalkyl group substitution, an alkylammonium group substitution, a nitrile group substitution, a phosphine group substitution, and a sugar group substitution. Modified cyclodextrins may include, but are not limited to methyl cyclodextrins (e.g., methyl β-cyclodextrin), hydroxyethyl cylcodextrins e.g., hydroxyethyl β-cyclodextrin) 2-hydroxypropyl cyclodextrins (e.g., 2-hydroxypropyl β-cyclodextrin and 2-hydroxypropyl γ-cyclodextrin), sulfobutyl cyclodextrins, glucosyl cyclodextrins (e.g., glucosyl α-cyclodextrin and glucosyl β-cyclodextrin), and maltosyl cyclodextrins (e.g., maltosyl α-cyclodextrin and maltosyl β-cyclodextrin).

The disclosed composition materials may be formed from ionic liquid compositions, for example, ionic liquid compositions comprising the one or more polysaccharides dissolved in one or more ionic liquids and preferably the one or more macrocyclic compounds dissolved in one or more ionic liquids. Suitable ionic liquids for forming the ionic liquid compositions may include but are not limited to alkylated imidazolium salts. In some embodiments, the alkylated imidazolium sail is selected from a group consisting of 1-butyl-3-methylimidazolium salt, 1-ethyl-3-methylimidazolium salt, and 1-allyl-3-methylimidazolium salt. Suitable salts may include, but are not limited to chloride salts.

In the disclosed ionic liquid compositions, a structural polysaccharide may be dissolved in an ionic liquid. In some embodiments, the ionic liquid may comprise at least about 2%, 4%, 6%, 8%, 10%, 15%, 20% w/w, dissolved structural polysaccharide.

In the disclosed ionic liquid compositions, a macrocyclic compound may be dissolved in the ionic liquid. In some embodiments, the ionic liquid may comprises at least about 2%, 4%, 6%, 8%, 10%, 15%, 20% w/w, dissolved macrocyclic compound.

The disclosed ionic liquid compositions may be utilized in methods for preparing the disclosed composite materials that comprise a structural polysaccharide and preferably a macrocyclic compound. For example, in the disclosed methods, a composite material comprising a structural polysaccharide and preferably a macrocyclic compound may be prepared by: (1) obtaining or preparing an ionic liquid composition as disclosed herein comprising a structural polysaccharide and preferably a macrocyclic compound, where the structural polysaccharide and preferably the macrocytic compound are dissolved in an ionic liquid; and (2) removing the ionic liquid from the ionic liquid composition and retaining the structural polysaccharide and preferably the macrocyclic compound. The ionic liquid may be removed from the compositions by steps that include, but are not limited to washing (e.g., with an aqueous solution). The water remaining in the composite materials after washing may be removed from the composite materials by steps that include, but are not limited to drying (e.g., in air) and lyophilizing (i.e., drying under a vacuum). The composite material may be formed into any desirable shape, for example, a film or a powder (e.g., a powder of microparticles and/or nanoparticles).

The disclosed composite materials may be utilized in a variety of processes. In some embodiments, the composite materials may be utilized to remove a contaminant from a stream (e.g., a liquid stream or a gas stream). As such, the methods may include contacting the stream with the composite material and optionally passing the stream through the composite material. Contaminants may include, but are not limited to, chlorophenols e.g., 2-chlorophenol, 3-chlorophenol, 4-chlorophenol, 3,4-dichlorophenol, and 2,4,5-triochlorophenol), bisphenol A, 2,4,6-trichloroanisole (e.g., as “cork taint” in wine), 1-methylocyclopropene, and metal ions (e.g., Cd²⁺, PB²⁺, and Zn²⁺).

In other embodiments, the composite materials may be utilized to remove toxins from an aqueous environment, for example, as part of a filter treatment or as part of a batch treatment. For example, the composite material may be contacted with toxins in water whereby the toxins have an affinity for the composite material and the toxins are incorporated into the composite material thereby removing the toxins from the water. Toxins removed by the disclosed methods may include any toxins that have an affinity for the composite material, which may include bacterial toxins such as microcystins which are produced by cyanobacteria. After the composite material has been utilized to remove toxins from the aqueous environment, the composite material may be regenerated by treating the composite material in order to remove the toxins from the composite material and enable the composite material to be reused again (i.e., via regeneration of the composite's capacity for adsorbing toxins).

In other embodiments, the composite material may be utilized to purify a compound (e.g., from an aqueous solution, a liquid stream, or a gas stream). For example, the composite material may be utilized to purify a compound from an aqueous solution, a liquid stream, or a gas stream that comprises the compound by contacting the aqueous solution, the liquid stream, or the gas stream with the composite material where the composite material has an affinity for the compound to be purified. In some embodiments, the compound may be purified from a mixture of compounds in an aqueous solution, a liquid stream, or a gas stream, for example where the composite material had a greater affinity for the compound to be purified than for the other compounds in the mixture. The composite material may be contacted with the aqueous solution, the liquid stream, or the gas stream comprising the mixture of compounds in order to bind preferentially the compound to be purified to the composite material and remove the compound from the mixture of compounds in the aqueous solution, the liquid stream, or the gas stream. In some embodiments, the compound to be purified is a specific enantiomer of the compound present in a racemic mixture of the compound, for example, where the composite material has a greater affinity for one enantiomer of the compound versus another enantiomer of the compound.

In other embodiments, the composite materials may be utilized to kill or eliminate microbes, including but not limited to bacteria. For example, the composite material may be contacted with bacteria including but not limited to Staphylococcus aureus (including methicillin-resistant strains), and Enterococcus faecalis (including vancomycin-resistant strains), Pseudomonas aeruginosa, Escherichia coli, in order to kill or eliminate the bacteria. The bacteria may be present in an aqueous solution, a liquid stream, or a gas stream as contemplated herein.

In other embodiments, the composite material may be utilized to inhibit the attachment and biofilm formation in water of various microbes including but not limited to bacteria such as Pseudomonas aeruginosa, Escherichia coli, Staphylococcus aureus, methicillin resistant S. aureus and vancomycin resistant Enterococcus faecalis. For example, where a substrate is utilized in an aqueous environment, the substrate may be coated with the composite material in order to inhibit or prevent bacterial growth and biofilm formation on the substrate

In other embodiments, the composite materials may be utilized to catalyze a reaction. For example, the composite materials may be utilized to catalyze a reaction by contacting a reaction mixture with the composite materials and optionally passing the reaction mixture through the composite material.

In other embodiments, the composite materials may be utilized to carry and release a compound. For example, the composite materials may be utilized to early and release a compound gradually over an extended period of time (e.g., a drug or a compound such as 1-methylocyclopropene in order to delay ripening of fruit or freshness of flowers). As such, the composite material may be utilized in packaging for fruit or flowers.

Preferably, a macro-cyclic compound is bound to the structural polysaccharide in the disclosed composite materials. As such, in the disclosed methods, preferably the macrocyclic compound is not removed from the composite material after a stream or a reaction mixture is contacted with the composite material or passed through the composite material.

The composite materials may be configured for a variety of applications. These include, but are not limited to, filter material for use in filters for liquid or gas streams, and fabric material for use in bandages for wounds or packaging for fruit or flowers.

### BRIEF DESCRIPTION OF THE DRAWINGS

FIG. 1. X-ray powder diffraction spectra of [BMIm⁺Cl⁻]; CS powder; α-TCD, β-TCD, and γ-TCD powder; and (A) [CS+α-TCD], (B) [CS+β-TCD], and (C) [CS+γ-TCD] composite materials at different stages of synthesis.

FIG. 2 (A) FTIR and (B) NIR spectra of 100% CS, α-TCD, and 50:50 CS/α-TCD composite material.

FIG. 3 SEM images of the surface (images on left column) and cross section (images on right column) of (A) 100% CEL, (B) 100% CS, (C) 50:50 CEL/γ-TCD, (D) 50:50 CEL/β-TCD, (E) 50:50 CS/γ-TCD, and (F) 50:50 CS/β-TCD.

FIG. 4 Plot of tensile strength as a function of γ-TCD concentration in [CEL+γ-TCD] composites and [CS+γ-TCD] composites.

FIG. 5 Intraparticle pore diffusion model plots for (A) 50:50 CS/β-TCD and (B) 50:50 CEL/β-TCD.

FIG. 6. Plot of equilibrium sorption capacity (qₑ) of all analytes by (A) 100% CEL and 100% CS, (B) 100% CEL and 50:50 CEL/β-TCD, (C) 100% CS and 50:50 CEL/β-TCD, and (D) all four composites.

FIG. 7. Plot of (A) qₑ and k for the adsorption of 2,4,5-trichlorophenol as a function of CS concentration in [CEL+CS] composite materials. (B) Sorption profiles of 50:50 CS/α-TCD, 50:50 CS/β-TCD, and 50:50 γ-TCD CS composites for 3,4-di-Cl-Ph. (C) Equilibrium sorption capacity for 3,4-dichlorophenol by CS+TCD composite materials as a function of α-TCD, β-TCD, and γ-TCD concentrations in the composites.

FIG. 8. Fitting of experimental values to the Langmuir, Freundlich, and Dubinin-Radushkevich isotherm models for the adsorption of 3,4-di-Cl-Ph onto the 50:50 CS/γ-TCD composite material.

FIG. 9. FTIR spectra of (A) 100% CS, β-TCD powder and 50:50 CS:β-TCD and (B) 100% CS, γ-TCD and 50:50 CS:γ-TCD.

FIG. 10. NIR spectra of (A) 100% CS, β-TCD powder and 50:50 CS:β-TCD and (B) 100% CS, γ-TCD and 50:50 CS:γ-TCD.

FIG. 11. A) FT-IR and B) NIR spectra of CEL/TCD composite materials.

FIG. 12. Pseudo second order linear plots for A) 100% CS and B) 1.00% CEL composite materials.

FIG. 13. Pseudo second order linear plots for A) 50:50 CS:β-TCD and B) 50:50 CEL:β-TCD composite materials.

### DETAILED DESCRIPTION

The disclosed subject matter further may be described utilizing terms as defined below.

Unless otherwise specified or indicated by context, the terms “a”, “an”, and “the” mean “one or more.” For example, “a compound” should be interpreted to mean “one or more compounds.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Disclosed are composite materials and ionic liquid compositions for preparing the composite materials. The composite materials typically include one or more structural polysaccharides and preferably one or more macrocyclic compounds.

As used herein, “structural polysaccharides” refer to water insoluble polysaccharides that may form the biological structure of an organism. Typically, structurally polysaccharides are polymers of 6-carbon sugars such as glucose or modified forms of glucose (e.g., N-acetylglucosamine and glucosamine), which are linked via beta-1,4 linkages. Structural polysaccharides may include, but are not limited to cellulose, chitin, and chitosan, which may be formed from chitin by deacetylating one or more N-acetylglucosamine monomer units of chitin via treatment with an alkali solution (e.g., NaOH). Chitosan-based polysaccharide composite materials and the preparation thereof are disclosed in Tran et al., J. Biomed. Mater. Res. Part A 2013:101A:2248-2257 (hereinafter “Tran et al. 2013), which is incorporated herein by reference.

As used herein, a “macrocyclic compound” is a cyclic macromolecule or a macromolecular cyclic portion of a molecule (e.g., a molecule including a ring of nine or more atoms, preferably including two or more potential donor atoms that may coordinate to a ligand). Macrocyclic compounds may include, but are not limited to cyclodextrins e.g., α-cyclodextrins, β-cyclodextrins, and γ-cyclodextrins), calixarenes, carcerands, crown ethers, cyclophanes, cryptands, cucurbiturils, pillararenes, and spherands.

The chemistry of macrocyclic compounds is of particular interest because these compounds (also known as “host compounds”) can entrap other molecules (known as a “guest compounds”) into their cavity to form an “inclusion complex.” A guest molecule can only be entrapped in the cavity of a macrocyclic compound if the guest molecule's size and shape are comparable to that of the cavity of the host compound. Therefore, a properly configured macrocyclic compound can selectively extract a guest compound from a mixture of many different compounds. As a consequence, macrocyclic compounds have been used in variety of applications including selective removal of contaminants and carriers of compounds such as drugs. Because the selectivity of macrocyclic compounds is dependent on the size and shape of its cavity, different types of macrocyclic compounds (cyclodextrins, calixarenes cucurbiturils, pillararenes and crown ethers) have different selectivity for different types of guest compounds. Of these, cyclodextrins are the only known macrocyclic compounds that are naturally occurring compounds. That is, they are completely biocompatible and biodegradable when used as a component of the presently disclosed composite materials. Other macrocyclic compounds (calixarenes, cucurbiturils, pillarenes and crown ethers) are all man-made compounds. In principle, they can be synthesized with relatively lower cost than the cost of cyclodextrins, and also they have different selectivity compared to those of cyclodextrins (e.g., some of them can form inclusion compounds with heavy metal ions).

The disclosed composite materials may be prepared from ionic liquid compositions that comprise one or more structural polysaccharides (and preferably one or more macrocyclic compounds) dissolved in one or more ionic liquids. As used herein, an “ionic liquid” refers to a salt in the liquid state, typically salts Whose melting point is less than about 100° C. Ionic liquids may include, but are not limited to salts based on an alkylated imidazolium cation, for example,

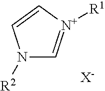

where R¹ and R² are C1-C6 alkyl (straight or branched), and X⁻ is any cation (e.g., a halide such as chloride, a phosphate, a cyanamide, or the like).

The disclosed composite materials may be utilized in methods for removing contaminants from aqueous solutions, liquid streams, or air streams. Chitosan-cellulose composite materials for removing microcystin are disclosed in Tran et al., J. of Hazard. Mat. 252-253 (2013) 355-366, which is incorporated herein by reference in its entirety.

The disclosed composite materials may be utilized in methods for purifying compounds from aqueous solutions, liquid streams, or air streams. In particular, the composite materials may be utilized in methods for purifying compounds from mixtures of compounds. Methods of using a chitosan-cellulose composite material for purifying a specific enantiomer of an amino acid from a racemic mixture are disclosed in Duri et al. Langmuir, 2014, 30 (2), pp 642-650 (hereinafter “Duri et al. 2014”), which is incorporated herein by reference in its entirety. As disclosed in Duri et al. 2014, in methods for purifying an enantiomer of a compound from a racemic mixture of a compound, the composite material may consist of structural polysaccharides (e.g., chitosan and cellulose). As such, the presence of a macrocyclic compound within the composite material may be optional where the composite material is utilized in methods for purifying an enantiomer of a compound from a racemic mixture of a compound.

The disclosed composite materials may be utilized in methods for inhibiting or preventing growth of microbes (e.g., bacteria). For example, the disclosed composite materials may be contacted with an aqueous solution, a liquid stream, or an air stream comprising microbes to inhibit or prevent growth of microbes in the aqueous solution, the liquid stream, or the air stream. Alternatively, the disclosed composite materials may be used to coat a substrate in order to inhibit or prevent growth of microbes on the substrate. The antimicrobial properties of chitosan-based polysaccharide composite materials are disclosed in Tran et al., J. Biomed. Mater. Res. Part A 2013:101A:2248-2257 (hereinafter “Tran et al. 2013) and Harkins A L, Duri S, Kloth L C, Tran C D. 2014. “Chitosan-cellulose composite for wound dressing material. Part 2. Antimicrobial activity, blood absorption ability, and biocompatibility.” J Biomed Mater Res Part B 2014: 00B: 000-000 (hereinafter “Harkins et al. 2014”), which are incorporated herein by reference in their entireties. As disclosed in Tran et al. 2013 and Harkins et al. 2014, in methods of using the disclosed composite materials for inhibiting or preventing microbial growth, the composite material may consist of structural polysaccharides (e.g., chitosan and cellulose). As such, the presence of a macrocyclic compound within the composite material may be optional where the composite material is utilized in methods for inhibiting or preventing microbial growth.

### EXAMPLES

The following examples are illustrative and are not intended to limit the claimed subject matter.

Reference is made to Duri et al., “Supramolecular Composition Materials from Cellulose, Chitosan, and Cyclodextrins: Facile Preparation and Their Selective Inclusion Complex Formation with Endocrine Disruptors,” Langmuir. 2013. 29 (16):5037-49, available on-line on Mar. 21, 2013; the content of which is incorporated herein by reference in its entirety.

### Abstract

We have successfully developed a simple and one step method to prepare high performance supramolecular polysaccharide composites from cellulose (CEL), chitosan (CS) and (2,3,6-tri-O-acetyl)-α-, β- and γ-cyclodextrin (α-, β- and γ-TCD). In this method, [BMIm⁺Cl⁻], an ionic liquid (IL), was used as a solvent to dissolve and prepare the composites. Since majority (>88%) of the IL used was recovered for reuse, the method is recyclable. XRD, FT-IR, NIR and SEM were used to monitor the dissolution process and to confirm that the polysaccharides were regenerated without any chemical modifications. It was found that unique properties of each component including superior mechanical properties (from CEL), excellent adsorbent for pollutants and toxins (from CS) and size/structure selectivity through inclusion complex formation (from TCDs) remain intact in the composites. Specifically, results from kinetics and adsorption isotherms show that while CS-based composites can effectively adsorb the endocrine disruptors (polychlorophenols, bisphenol-A), its adsorption is independent on the size and structure of the analytes. Conversely, the adsorption by γ-TCD-based composites exhibits strong dependency on size and structure of the analytes. For example, while all three TCD-based composites (i.e., α-, β- and γ-TCD) can effectively adsorb 2-, 3- and 4-chlorophenol, only γ-TCD-based composite can adsorb analytes with bulky groups including 3,4-dichloro- and 2,4,5-trichlorophenol. Furthermore, equilibrium sorption capacities for the analytes with bulky groups by γ-TCD-based composite are much higher than those by CS-based composites. Together, these results indicate that γ-TCD-based composite with its relatively larger cavity size can readily form inclusion complexes with analytes with bulky groups, and through inclusion complex formation, it can strongly adsorb much more analytes and with size/structure selectivity compared to CS-based composites which can adsorb the analyte only by surface adsorption.

### Introduction

Supramolecular composite material is an organized, complex entity that is created from the association of two or more chemical species held together by various intermolecular forces.¹⁻⁵ Its structure is the result of not only additive but also cooperative interactions, and its properties are often better than the sum of the properties of each individual component.¹⁻³ Supramolecular composite materials containing marcrocyclic polysaccharides such as cyclodextrins (CDs) are of particular interest because CD ((α-, β- and γ-CD) are known to form selective inclusion complexes with a variety of different compounds with different sizes and shapes.⁴⁻⁶ To be able to fully and practically utilize properties of CD-based supramolecular composite material, it is necessary for the materials to be readily fabricated in solid form (film and/or particle) in which encapsulated CDs fully retain their unique properties. CDs are highly soluble in water, and cannot be processed in film because of its poor mechanical and theological strength. As a consequence, it is often necessary to chemically react and/or graft CD onto man-made polymers to increase its mechanical strength so that the resultant materials can be processed into solid thin film and/or particles:⁷⁻¹⁰ CD-based materials synthesized by these methods have been reported. Unfortunately, in spite of their potentials, practical applications of such materials are rather limited because in addition to complexity of reactions used in the synthesis which are limited to persons with synthetic expertise, method used may also alter and/or lessen desired properties of CDs.^{7,8,11,12} It is, therefore, desirable to improve the mechanical strength of CD-based supramolecular material so that it can be fabricated into a solid film for particles) not by chemical modification with synthetic chemicals and/or polymers but rather by use of naturally occurring polysaccharides such as cellulose and/or chitosan which are structurally similar to CDs.

Cellulose (CEL) and chitosan (CS) are two of the most abundant biorenewable biopolymers on the earth. The latter is derived by N-deacetylation of chitin which is the second most abundant naturally occurring polysaccharide found in the exoskeletons of crustaceans such as crabs and shrimp. In these polysaccharides, an extensive network of intra- and inter-hydrogen bonds enables them to adopt an ordered structure. While such structure is responsible for CEL to have superior mechanical strength and CS to exhibit remarkable properties such as hemostasis, wound healing, bactericide and fungicide, drug delivery and adsorbent for organic and inorganic pollutants, it also makes them insoluble in most solvents.^{9,10,13-18} This is rather unfortunate because with their superior mechanical strength and unique properties, CEL and CS would be excellent supporting polymer for CD. It is expected that the resulting [CEL and/or CS+CD] composite would have properties that are a combination of those of all of its components. That is, it may have superior mechanical strength (from CEL), can stop bleeding, heal wound, kill bacteria, deliver drugs (from CS) and selectively form inclusion complexes with a wide variety of compounds of different types, sizes and shapes (from CDs). Unfortunately, to date, such supermolecules have not been realized because of lack of a suitable solvent which can dissolve all three compounds. The difficulty stems from the fact that while CDs are water soluble CEL and CS are insoluble in most solvents. Furthermore, there is not a solvent or system of solvents which can dissolve all three CEL. CS and CD.

Considerable efforts have been made in the last few years to find suitable solvents for CEL and CS, and several solvent systems have been reported.^{19,20} For example, high temperature and strong exotic solvents such as methylmorpholine-N-oxide, dimethylthexylsilyl chloride or LiCl in dimethylacetamide (DMAc) are needed to dissolve CEL whereas an acid such as acetic acid is required to protonate amino groups of CS so that it can be dissolved in water.⁶⁻²⁹ These methods are undesirable because they are based on the use of corrosive and volatile solvents, require high temperature and suffer from side reactions and impurities which may lead to changes in structure and properties of the polysaccharides. More importantly, it is not possible to use a single solvent or system of solvents to dissolve both CEL and CS. A new method which can effectively dissolve all three CS. CEL and CD, not at high temperature and not by corrosive and volatile solvents but rather by recyclable “green” solvent is particularly needed. This is because such method would facilitate preparation of [CS+CD] and [CEL+CD] composite materials which are not only biocompatible but also have combined properties of its components.

Recently, we have developed a new method which can offer a solution for this problem.²¹ In this method, we (1) exploited advantages of a simple ionic liquid, butyl methylimmidazolium chloride (BMIm⁺Cl⁻), a green solvent,²²⁻²⁵ to develop an innovative, simple, pollution-five method to dissolve not only CS but also other polysaccharides including CEL without using any acid or base, thereby avoiding any possible chemical or physical changes, and (2) used only naturally occurring biopolymers such as CEL as support materials to strengthen structure and expand utilities while keeping the biodegradable, biocompatible and anti-infective and drug carrier properties of CS-based materials intact. Using this method, we have successfully synthesized composite materials containing CEL and CS with different compositions. As expected, the composite materials obtained were found to have combined advantages of their components, namely superior chemical stability and mechanical stability from CEL) and excellent antimicrobial properties (from CS). The [CEL+CS] composite materials inhibit growth of a wider range of bacteria than other CS-based materials prepared by conventional methods. Specifically, it was found that over a 24 hr period, the composite materials substantially inhibited growth of bacteria such as Methicillin Resistant Staphylococcus Aureus (MRSA), Vancomycin Resistant Enterococcus (VRE), S. aureus and E. coli.²¹

The information presented is indeed provocative and clearly indicate that it is possible to use this simple, one-step process without any chemical modification to synthesize novel supramolecular composite materials from CEL, CS and CDs. Based on results of our previous work on the [CEL+CS] composites²¹, it is expected that the [CEL and/or CS+CD] composite materials may possess all properties of their components, namely mechanical strength (from CEL), excellent adsorbent for toxins and pollutants (from CS and selectively form inclusion complexes with substrates of different sizes and shapes (from CDs). Such considerations prompted us to initiate this study which aims to hasten the breakthrough by using the method which we have developed recently to synthesize novel supramolecular composite materials from CEL, CS and CDs. Results on the synthesis, spectroscopic characterization and applications of the composite materials for removal of organic pollutants such as endocrine disruptors are reported herein.

### Experimental Section

Chemicals. Cellulose (microcrystalline powder) and chitosan (MW≈310-375 kDa) were purchased from Sigma-Aldrich (Milwaukee, Wis.) and used as received. [BMIm⁺Cl⁻] was synthesized from freshly vacuum distilled 1-methylimidazole and 1-chlorobutane (Alfa Aesar, Ward Hill, Mass.) using procedure previously used in our lab.²¹⁻²⁶ 2-chlorophenol (2 Cl-Ph), 3 chlorophenol (3 Cl-Ph), 4-chlorophenol (4 Cl-Ph), 3,4 dichlorophenol (3,4 di Cl-Ph), 2,4,5 trichlorophenol (2,4,5 tri Cl-Ph) and bisphenol A (BPA) were from Sigma Aldrich (Milwaukee, Wis.). Heptakis(2.3,6-tri-O-acetyl)-β-cyclodextrin (β-TCD) (TCI America, Portland, Oreg.), hexakis(2,3,6-tri-O-acetyl)-α-cyclodextrin (α-TCD) and octakis(2,3,6-tri-O-acetyl)-γ-cyclodextrin (γ-TCD) (Cyclodextrin-Shop, The Netherlands) were used as received. Scheme 1 illustrates the structures of compounds used in this study.

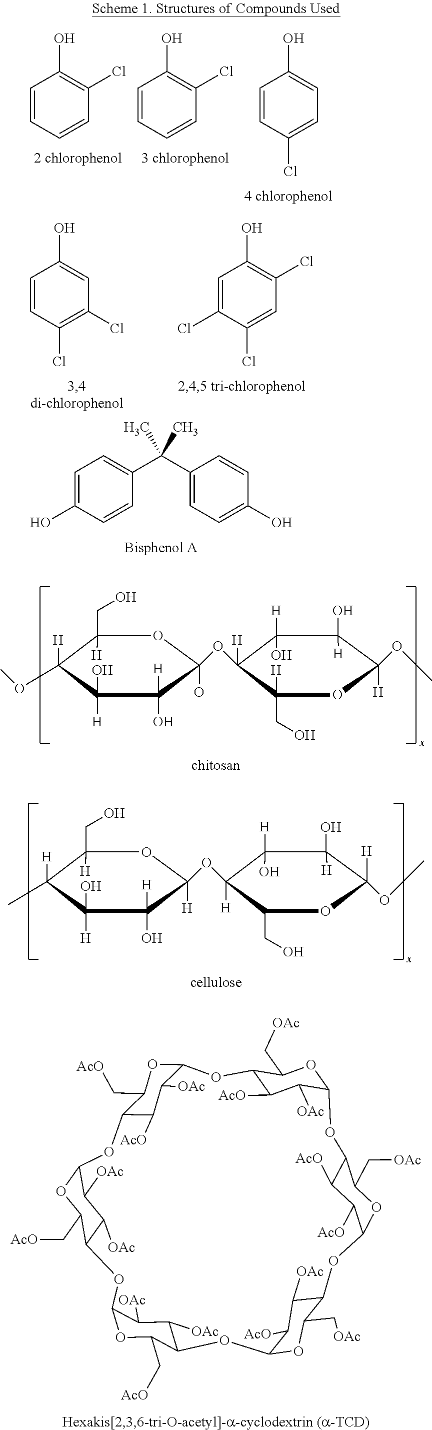

Instrumentation. Elemental analysis was carried out by Midwest Microlab, LLC (Indianapolis, Ind.). ¹H NMR spectra were taken on a VNMRS 400 spectrometer. Near-infrared (NIR) spectra were recorded on a home-built NIR spectrometer.²⁷ FT-IR spectra were measured on a PerkinElmer 100 spectrometer at 2 cm⁻¹ resolution with either KBr or by a ZnSe single reflection ATR accessory (Pike Miracle ATR). X-ray diffraction (XRD) measurements were taken on a Rigaku MiniFlex II diffractometer utilizing the Ni filtered Cu Kα radiation (1.54059 Å).²⁸ Scanning electron microscopic images of surface and cross section of the composite materials were taken under vacuum with an accelerated voltage of 3 kV using Hitachi S4800 scanning electron microscope (SEM). Tensile strength measurements were performed on an Instron 5500R Tensile Tester.

Preparation of [CEL+TCD] and [CS+TCD] Composite Films. As illustrated in Scheme 2, [CEL+α-TCD, β-TCD and γ-TCD] and [CS+α-TCD, β-TCD and γ-TCD] composite materials were synthesized using a procedure similar to that previously developed in our laboratory for the synthesis of CEL, CS and [CEL+CS].²

Essentially, as shown in Scheme 2, an ionic liquid. [BMIm⁺ Cl⁻], was used as a solvent to dissolve CEL, CS, α-TCD, β-TCD and γ-TCD. Dissolution was performed at 100° C. and under Ar or N₂ atmosphere. All polysaccharides were added in portions of approximately 1 wt % of the ionic liquid. Succeeding portions were only added after the previous addition had completely dissolved until the desired concentration has been reached. For composite films, the components were dissolved one after the other, with CEL (or CS) being dissolved first and TCDs last. Using this procedure, solutions of CEL (containing up to 10% w/w (of IL)), CS (up to 4% w/w) and composite solutions containing CEL (or CS) and α-TCD, β-TCD or γ-TCD with various proportions were prepared in about 6-8 hours.

Upon complete dissolution, the homogeneous solutions of the polysaccharides in [BMIm⁺Cl⁻] were cast on glass slides or Mylar sheets using a RDS stainless steel coating rod with appropriate size (RDS Specialties, Webster, N.Y.) to produce thin films with different compositions and concentrations of CEL (or CS) with α-TCD, β-TCD or γ-TCD. If necessary, thicker composite materials can be obtained by casting the solutions onto PTFE moulds of the desired thickness. They were then kept at room temperature for 24 hours to allow the solutions to undergo gelation to yield GEL Films. The [BMIm⁺ Cl⁻] remaining in the film was then removed by washing the films in deionized water for about 3 days to yield WET Films. During this period, the washing water was constantly replaced with fresh deionized water to maximize the removal of the ionic liquid. The [BMIm⁺ Cl⁻] used was recovered from the washed aqueous solution by distillation. It was found that at least 88% of [BMIm⁺Cl⁻] was recovered for reuse. The regenerated composite materials were lyophilized overnight to remove water, yielding dried porous composite films (DRY films).

Procedure Used to Measure Kinetics of Adsorption. Two matching cuvettes were used for all adsorption measurements, one for adsorption of the pollutant by the composite and the other as the blank (blank 1). The samples (about 0.02 g of dry film of the composite material) was washed thoroughly in water prior to the adsorption experiments to further insure that [BMIm⁺ Cl⁻] was completely removed because absorption of any residual IL may interfere with that of polychlorophenols or BPA. To wash the samples, the weighed composite materials were placed in a thin cell fabricated from PTFE whose windows were covered by two PTFE meshes. The meshes ensured free circulation of water through the material during the washing process, The PTFE mould containing the samples was placed in a 2 L beaker which was filled with de-ionized water and was stirred at room temperature for 24 hours. During this time, absorbance of washed water was monitored at 214 and 287 nm to determine the presence of any [BMIm⁺ Cl⁻]. The water in the beaker was replaced with fresh de-ionized water every 4 hours.

After 24 hours, the composite material was taken out of the water and placed into the sample cuvette. Both sample and blank cells were stirred using a small magnetic spin bar during the measurement. In order to prevent damage to the sample by the magnetic spin bar and to maximize the circulation of the solution during measurement, the samples were sandwiched between two PTFE meshes. Specifically, a piece of PTFE mesh was placed at the bottom of the spectrophotometric cell. The washed film sample was laid flat on top of the PTFE mesh. Another piece of PTFE mesh was placed on top of the sample and finally the small magnetic spin bar was placed on top of the second mesh. The blank cell had the same contents as the sample cell but without the composite material. Exactly 2.70 mL of 1.55×10⁻⁴M aqueous solution of polychlorophenol or BPA was added to both sample and blank cell. A second blank cell (blank 2) was also employed. This blank cell 2 had the same contents as the sample cell (i.e., PTFE mesh, composite film, PTFT mesh and magnetic spin bar) but without the pollutant. Any adsorption of the pollutants by the cell content (PEFE mesh, magnetic spin bar) and not by the composite materials was corrected by the signal of blank 1. Blank 2 provided information on any possible interference of absorption of pollutant by leakage of residual IL from the composite film. Measurements were carried out on a Perkin Elmer Lambda 35 UV/VIS spectrometer set to the appropriate wavelength for each pollutant, i.e., 274 nm for 2- and 3-chlorophenol, 280 nm for 4-chlorophenol, 282 nm and 289 nm for 3,4-dichloro- and 2,4,5-trichlorophenol, respectively, and 276 nm for bisphenol A. Measurements were taken at 10 minute intervals during the first 2 hours and 20 minute intervals after 2 hours. After each measurement, the cell was returned to a magnetic stirrer for continuous stirring. Reported values were the difference between the sample signals and those of blank1 and blank2. However, it was found that signals measured by both blank cells were negligible within experimental error.

Analysis of Kinetic Data. The pseudo-first-order, pseudo-second-order and intra-particle diffusion kinetic models were used to evaluate the adsorption kinetics of different polychlorophenols and BPA and to quantify the extent of uptake in the adsorption process.

Pseudo-first-order kinetic model. The linear form of Lagergren's pseudo-first-order equation is given as:⁵²

ln(qₑ−qₜ)=ln qₑ−k₁t [SI-1]

where qₜ and qₑ are the amount of pollutant adsorbed at time t and at equilibrium (mg) g⁻¹) respectively and k₁ (min⁻¹) is the pseudo first order rate constant calculated from the slope of the linear plot of ln (qₑ−qₜ) versus t.

Pseudo-second-order kinetic model. According to the Ho model, the rate of pseudo second order reaction may be dependent on the amount of species on the surface of the sorbent and the amount of species sorbed at equilibrium. The equilibrium sorption capacity, qₑ, is dependent on factors such as temperature, initial concentration and the nature of solute-sorbent interactions. The linear expression for the Ho model can be represented as follows:⁵²

$$\frac{t}{{q}_{t}} = \frac{1}{{k}_{2} {q}_{e}^{2}} + \frac{1}{{q}_{e}} t \qquad [ SI \text{-} 2 ]$$

where k₂ is the pseudo-second order rate constant of sorption (g/mg.min), qₑ is the amount of analyte adsorbed at equilibrium (mg/g), qₜ is the amount of analyte adsorbed at any time t (mg/g).

If the initial adsorption rate h is

h=k₂qₑ² [SI-3]

Then Eq SI-2 can be rearranged as

$$\frac{t}{{q}_{t}} = \frac{1}{h} + \frac{1}{{q}_{e}} t \qquad [ SI \text{-} 4 ]$$

A linear plot can be obtained by plotting t/qₜ against t. qₑ and h, can obtained from the slope and intercept; k₂ can be calculated from h and qₑ according to Eq SI-3.

Intra-particle diffusion model. The intra-particle diffusion equation is given as follows:^{51,53}

qₜ=kᵢt^{0.5}+I [SI-5]

where kᵢ (mg g⁻¹ min^{-0.5}) is the intra-particle diffusion rate constant and I (mg g⁻¹) is a constant that gives the information regarding the thickness of the boundary layer.^{51,53} According to this model, if the plot of qt versus t^{0.5} gives a straight line, then the adsorption process is controlled by intra-particle diffusion, while, if the data exhibit multi-linear plots, then two or more steps influence the adsorption process.

Procedure Used to Measure Equilibrium Sorption Isotherms. Batch sorption experiments were carried out in 50 mL stoppered vials containing 10 mL of the pollutant solution of known initial concentration. A weighed amount (0.1 g) of the composite material was added to the solution. The samples were agitated at 250 rpm in a shaking water bath at 25° C. for 72 hours. The residual amount of pollutant in each flask was analyzed by UV/Vis spectrophotometry. The amount of pollutant adsorbed onto the composite material was calculated using the following mass balance equation:

$${q}_{e} = \frac{( {C}_{i} - {C}_{e} ) V}{m} \qquad [ 1 ]$$

where qₑ (mg/g) is the equilibrium sorption capacity, Cᵢ and Cₑ (mg/L) are the initial and final pollutant concentrations respectively. V (L) is the volume of the solution and m (g) is the weight of the composite film material.

Analysis of Adsorption Isotherms. Different isotherm models have been developed for describing sorption equilibrium. The Langmuir, Freundlich and Dubinin-Raduslikevich (D-R) isotherms were used in the present study.

Langmuir isotherm. The Langmuir sorption isotherm describes that the uptake occurs on a homogeneous surface by monolayer sorption without interaction between adsorbed molecules and is commonly expressed as (Langmuir, 1916):⁵⁴

$$\frac{{C}_{e}}{{q}_{e}} = \frac{{C}_{e}}{{q}_{m}} + \frac{1}{{K}_{L} {q}_{m}} \qquad [ SI \text{-} 6 ]$$

where qₑ (mg g⁻¹) and Cₑ (mg L⁻¹) are the solid phase concentration and the liquid phase concentration of adsorbate at equilibrium respectively, qₘ (mg g⁻¹) is the maximum adsorption capacity, and K_{L} (L mg⁻) is the adsorption equilibrium constant. The constants K_{L} and qₘ can be determined from the slope and intercept of the plot between Cₑ/qₑ and Cₑ.

Freundlich isotherm. The Freundlich isotherm is applicable to non-ideal adsorption on heterogeneous surfaces and the linear form of the isotherm can be represented as (Freundlich, 1906).⁵⁵

$$log {q}_{e} = log {K}_{F} + ( \frac{1}{n} ) log {C}_{e} \qquad [ SI \text{-} 7 ]$$

where qₑ (mg g⁻¹) is the equilibrium concentration on adsorbent, Cₑ (mg L⁻¹) is the equilibrium concentration in solution, K_{F} (mg g⁻¹) (L g⁻¹)^{1/n} is the Freundlich constant related to sorption capacity and n is the heterogeneity factor. K_{F} and 1/n are calculated from the intercept and slope of the straight line of the plot log qₑ versus log Cₑ. n value is known to be a measure of the favorability of the sorption process.⁵⁸ A value between 1 and 10 is known to represent a favorable sorption.

Dubinin-Radushkevich (D-R) isotherm. The Dubinin-Radushkevich (D-R) isotherm model envisages about the heterogeneity of the surface energies and has the following formulation:⁵⁷

$$\begin{matrix}ln {q}_{e} = ln {q}_{m} - {βɛ}^{2} & [ SI \text{-} 8 ] \\ ɛ = RT ln ( 1 + \frac{1}{{C}_{e}} ) & [ SI \text{-} 9 ]\end{matrix}$$

where qₘ (mg g⁻¹) is the maximum adsorption capacity, β (mmol² J⁻²) is a coefficient related to the mean free energy of adsorption, ε (J mmol⁻¹) is the Polanyi potential, R is the gas constant (8.314 J mol⁻¹ K⁻¹), T is the temperature (K) and Cₑ (mg L⁻¹) is the equilibrium concentration. The D-R constants qₘ and β can be determined from the intercept and slope of the plot between ln qₑ and ε².

The constant β in the D-R isotherm model is known to relate to the mean free energy E (KJ mol⁻¹) of the sorption process per mole of the analyte which in turn can give information about the sorption mechanism. E can be calculated using the equation 1 below.⁵⁹

$$E = \frac{1}{\sqrt{2} β} \qquad [ SI \text{-} 10 ]$$

According to this theory, the adsorption process is supposed to proceed via chemisorb if E is between 8 and 16 KJmol⁻¹ whereas for values less than 8 KJmol⁻¹, the sorption process is often governed by physical nature.)⁵⁹

### Results and Discussion

### Synthesis and Characterization of CEL/CS+α-TCD, β-TCD and γ-TCD Composite Materials

The CS used in this study was specified by the manufacturer (Sigma-Aldrich) as haying a degree of deacetylation (DA) value of 75%. As will be described below, because unique properties of CS including its ability to adsorb pollutants are due to its amino groups, experiments were performed to determine its DA value. Two different methods, FT-IR and ¹H NMR, were employed for the determination.³⁰⁻³⁵ For FT-IR method, the spectra were taken at 2 cm⁻¹ resolution. The CS sample was vacuum dried at 50° C. for 2 days. A small amount of the dried sample was then ground in tar and pressed into a pellet for FT-IR measurements. Four KBr pellets were prepared and their spectra were recorded. Degree of deacetylation (DA) was calculated from the four spectra, and average value is reported together with standard deviation. The DA value was calculated based on the following equation:³⁰⁻³¹

DA(%)=100−[(A₁₆₅₅/A₃₄₅₀)*100/1.33] [2]

where A₁₆₅₅ and A₃₄₅₀ are the absorbances at 1655 cm⁻¹ of the amide C═O and 3450 cm⁻¹ of the OH band respectively. The factor 1.33 denotes the value of the ratio of A₁₆₅₅/A₃₄₅₀ for fully N-acetylated chitosan. A DA% value of 84±2 was found using this method.

For ¹H NMR determination, the spectra were taken at 70° C. About 5 mg of chitosan sample which was previously vacuum dried at 50° C. for 2 days, was dissolved in 0.5 mL of 2 wt % DCl/D2O solution at 70° C. The degree of deacetylation (DA) was evaluated from the following equation using the integral intensity, I_{CH3}, of the CH₃ residue of N-acetyl, and the sum of the integral intensities, I_{H2-H6}, of protons 2-6 of the chitosan residue:³⁵

$$DA ( \% ) = [ 1 - ( \frac{1}{3} {I}_{{CH}_{3}} \text{/} \frac{1}{6} {I}_{H 2 - H 6} ) ] 100 \qquad [ 3 ]$$

A DA value of 78% was found using this method.

It has been reported that chitosan samples may contain some protein impurities. Accordingly, experiments were carried out to determine ally possible protein impurities in the CS sample used in this study. The percentage of proteins impurity (%P) can be calculated from the following equation:³⁶⁻³⁸

%P=(%N−N_{T})×6.25 [4]

where 6.25 corresponds to the theoretical percentage of nitrogen in proteins: %N represents the percentage of nitrogen measured by elemental analysis; N_{T} represents the theoretical nitrogen content of chitosan sample. It was calculated based on the degree of deacetylation (DA) of chitosan and percentage of nitrogen for fully acetylated chin and fully deacerylated chitosan (6.89 and 8.69),³⁶⁻³⁸ respectively. Using DA values of 84% (from FT-IR) and 78% (from NMR), percentage of protein impurities in CS sample were found to be 1.89%, and 1.24%, respectively. When errors associated with elemental analysis and with the determination of DA values by FT-IR and NMR method are taken into account, it can be assumed that these two %P values are the same within experimental errors.

As described in the Experimental Section, [BMIm⁺ Cl⁻] was used as the sole solvent to dissolve CEL, CS and TCD to prepare the [CEL+TCD] and [CS+TCD] composite materials. It is noteworthy to add that [BMIm⁺ Cl⁻] is not the only IL that can dissolve the polysaccharides. Other ILs including ethylmethylimidazolium acetate (EMIm⁺Ac⁻), BMIm⁺Ac⁻ and allylmethylimidazolium chloride (AMIm⁺ Cl⁻) are also known to dissolve the polysaccharides as well. [BMIm⁺ Cl⁻] was selected because compared to these ILs it can dissolve relatively higher concentration of the polysaccharides. For example, the solubility of CEL in [BMIm⁺ Cl⁻], [AMIm⁺Cl⁻] [BMIm⁺Ac⁻] and [EMIm⁺Ac⁻] was reported to be 20%, 12% and 8%, respectively). Furthermore, [BMIm⁺ Cl⁻] is relatively cheaper than these ILs because it can easily be synthesized in a one-step process from relatively inexpensive reagents (1-methylimidazole and 1-chlorobutane) whereas other ILs are relatively more expensive as they require more expensive reagents (silver acetate) and two-step synthetic process.^{29, 39-43}

Since [BMIm⁺ Cl⁻] is totally miscible with water, it was removed from the Gel Films of the composites by washing the films with water. Washing water was repeatedly replaced with fresh water until it is confirmed that there was no ILs in the washed water (by monitoring UV absorption of the IL at 214 nm and 287 nm). The IL used was recovered by distilling the washed aqueous solution (the IL remained because it is not volatile). The recovered [BMIm⁺Cl⁻] was dried under vacuum at 70° C. overnight before reuse. It was found that at least 88% of [BMIm⁺Cl⁻] was recovered for reuse. As such, the method developed here is recyclable because [BMIm⁺Cl⁻] is the only solvent used in the preparation and majority of it was recovered for reuse.

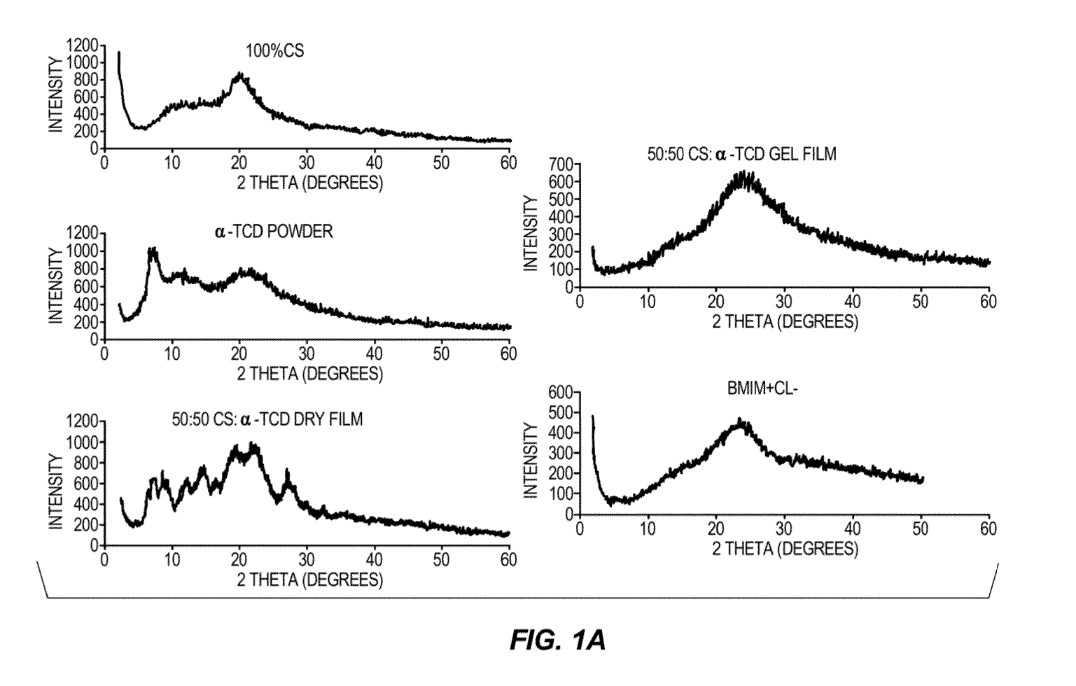

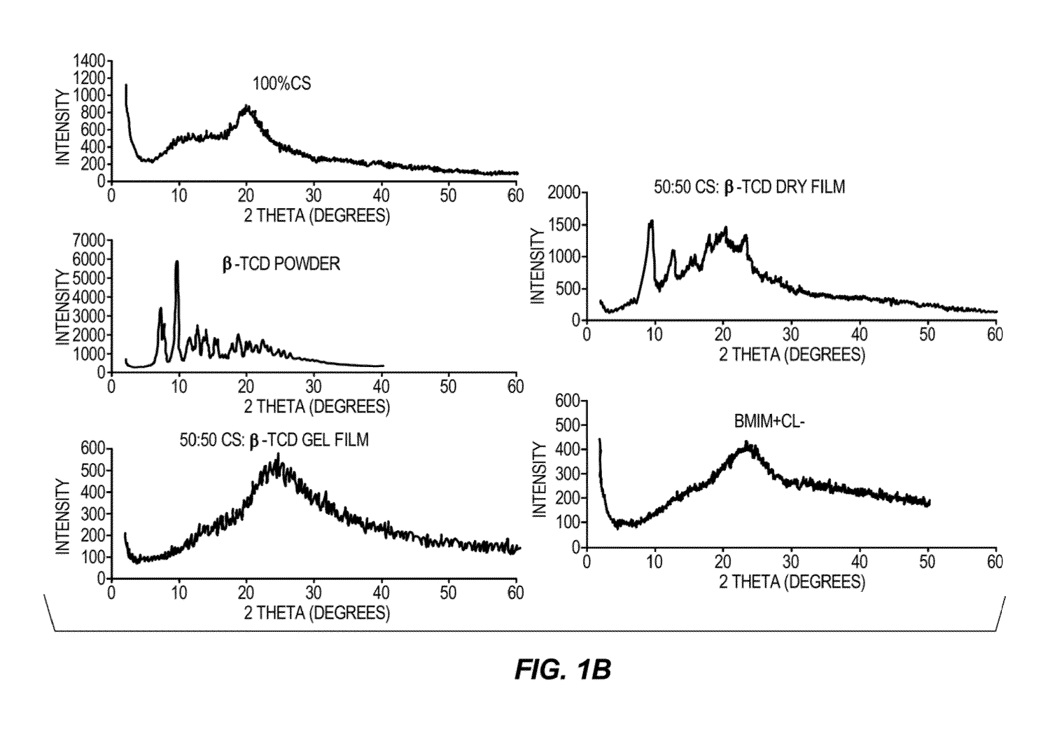

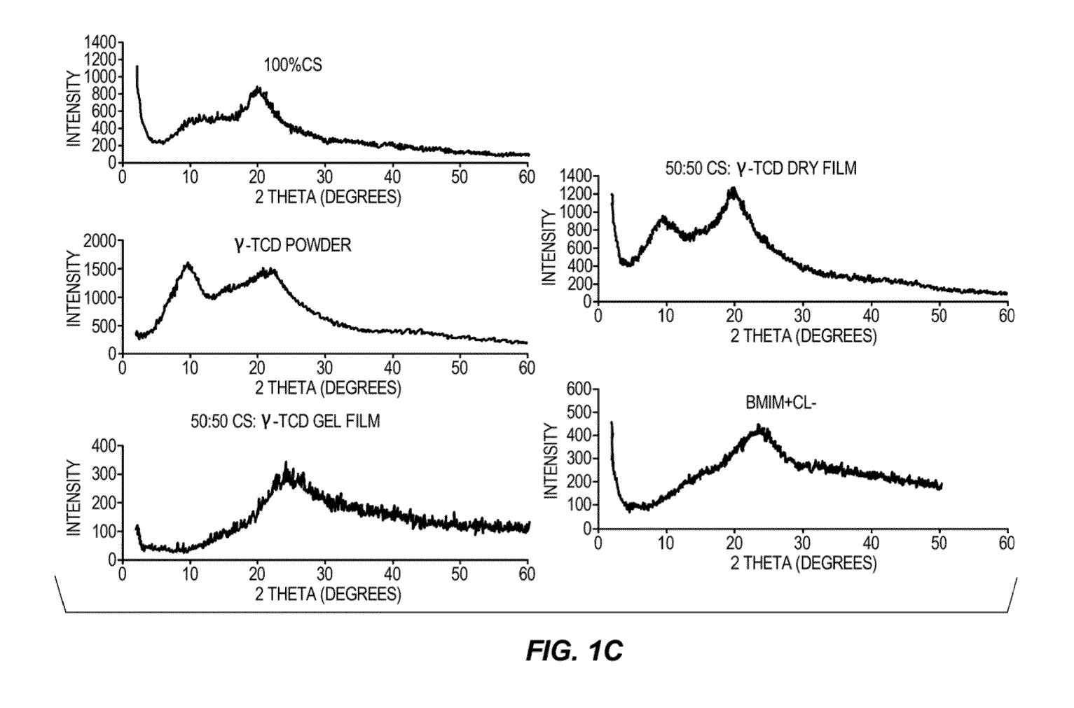

The dissolution of the polysaccharides, for example, CS and TCD, in [BMIm⁺Cl⁻] ionic liquid and their regeneration in the composite materials was followed and studied by powder X-ray diffraction (XRD). FIG. 1 shows the XRD spectra of the [CS+α-TCD], [CS+β-TCD] and [CS+γ-TCD] composites at various stages of preparation. Difference among XRD spectra of the α-, β- and γ-TCD materials (red curves in 1A, B and C, respectively) seems to indicate that these starting cyclodextrin materials have different structural morphologies. While the XRD spectrum of the β-TCD powder is consistent with a highly crystalline structure, the XRD spectra of α-TCD and γ-TCD seem to suggest that these CDs have an amorphous structure². The XRD spectra of [BMIm⁺Cl⁻] (black curves) and the gel films (purple curves) were measured to determine the dissolution of the CS and TCDs in the ionic liquid. As illustrated, the XRD spectra of the gel films are similar to that of [BMIm⁺Cl⁻], and do not exhibit any of the CS or TCD diffraction peaks. The absence of the XRD peaks of CS and TCDs and the similarity between the spectra of the gel films to that of the [BMIm⁺Cl⁻] clearly indicate that [BMIm⁺Cl⁻] completely dissolved CS and TCDs. The XRD spectra of the regenerated composite films (Dry films) also shown in FIG. 1. As expected, the XRD spectra of the 50:50 CS:α-TCD, 50:50 CS:β-TCD and 50:50 CS:γ-TCD regenerated composite films exhibit XRD peaks which can be attributed to those of α-TCD, β-TCD and γ-TCD respectively.

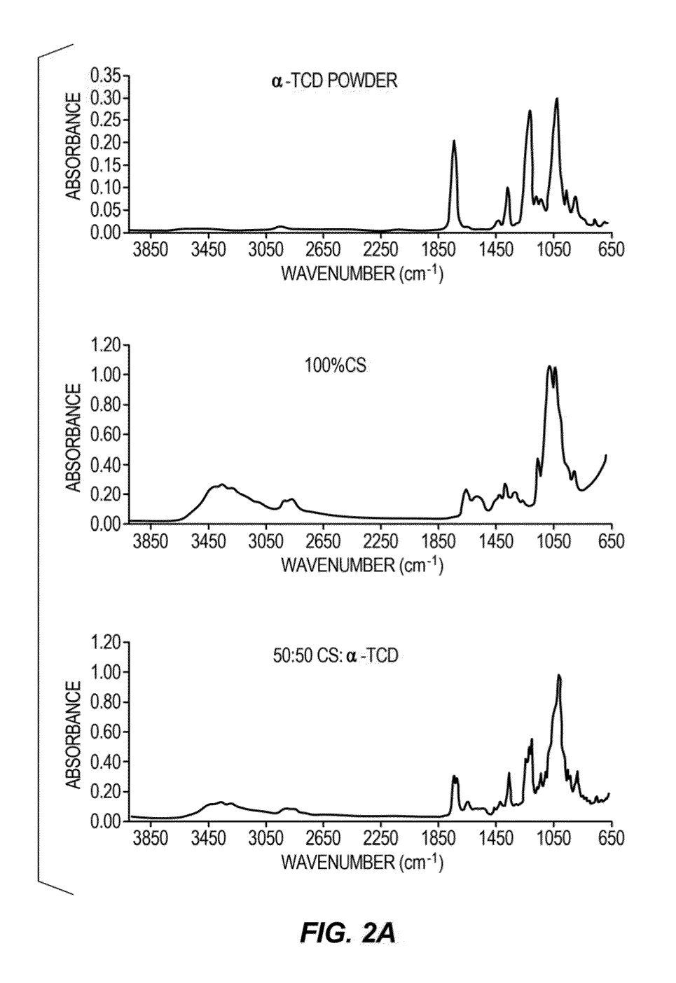

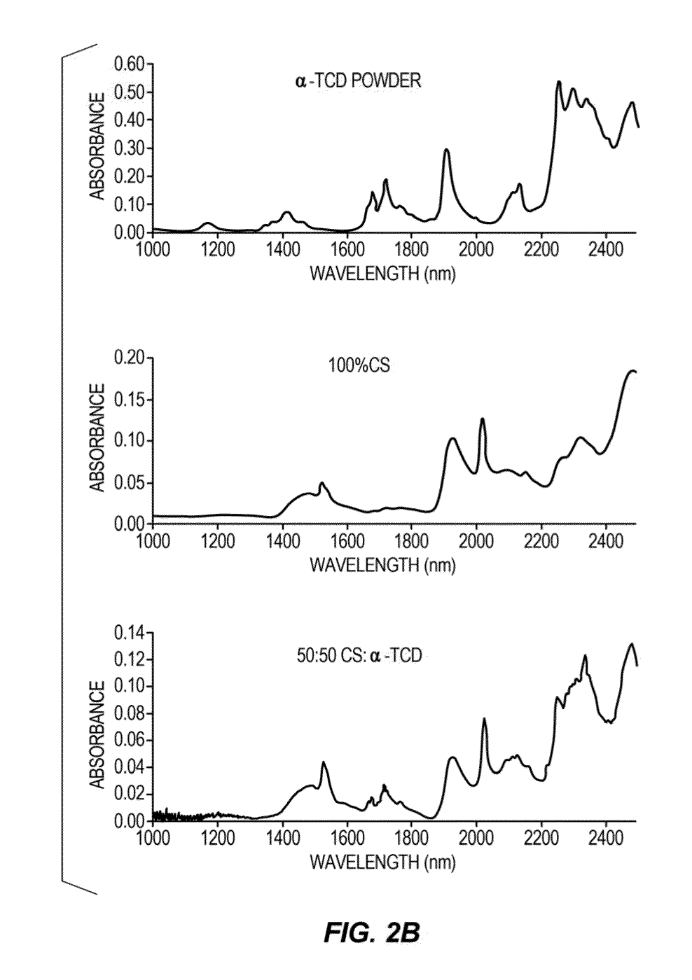

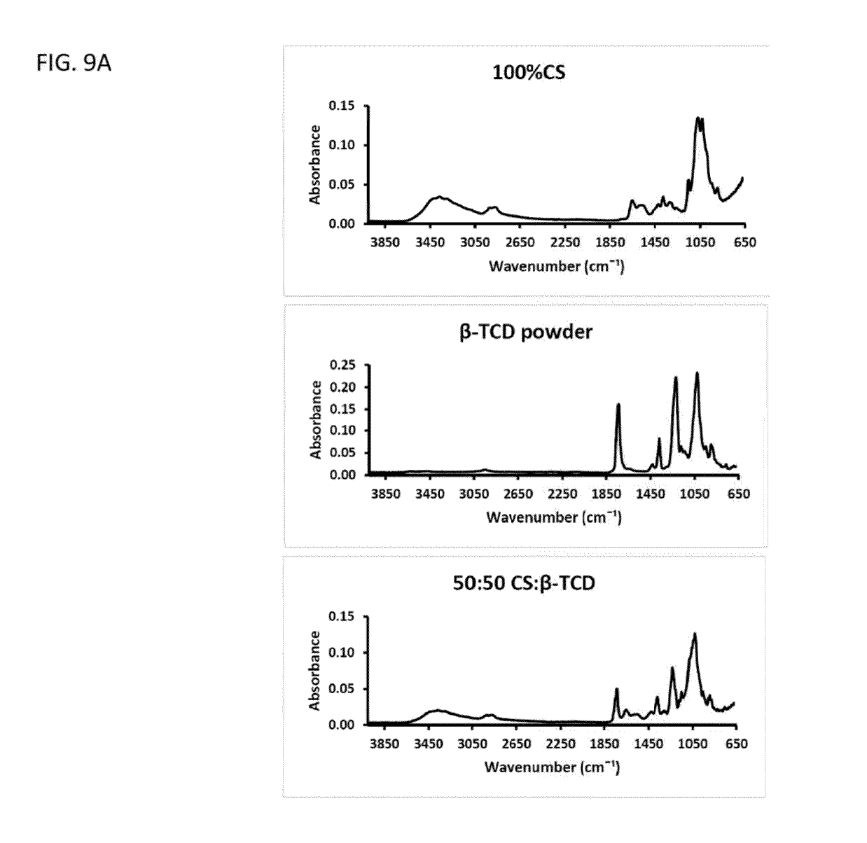

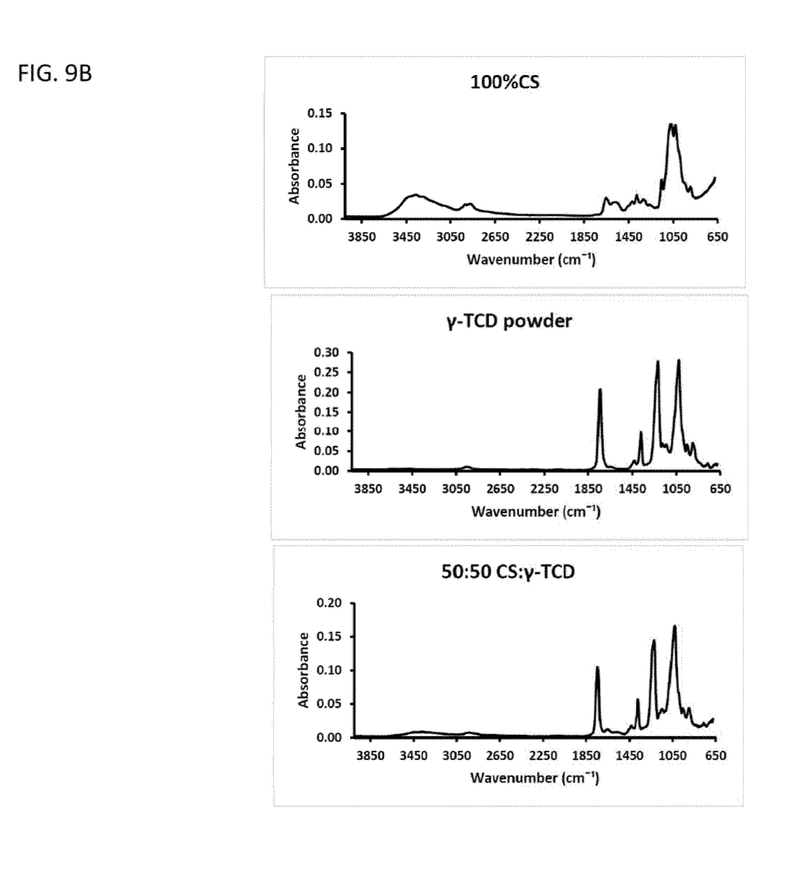

FT-IR and NIR spectroscopy was used to Characterize the chemical composition of the resultant composite films. The FT-IR and NIR spectra of the α-TCD powder, 100% CS and [CS+ α-TCD] composite materials are shown in FIGS. 2A and 2B, respectively (those corresponding to β-TCD and γ-TCD are shown in FIGS. 9&B and 10A&B). As illustrated, the FT-IR spectrum of a 100% CS Dried Film displays characteristic CS bands around 3400 cm⁻¹ (O—H stretching vibrations), 3250-3350 cm⁻¹ (symmetric and asymmetric N—H stretching), 2850-2900 cm⁻¹ (C—H stretching), 1657 cm⁻¹ (C═O, amide 1), 1595 cm⁻¹ (N—H deformation), 1380 cm⁻¹ (CH₃ symmetrical deformation), 1319 cm⁻¹ (C—N stretching, amide III) and 890-1150 cm⁻¹ (ether bonding).^{21,35-37} For reference, FT-IR spectrum of α-TCD starting material is also shown as red curve in 2A (and those β- and γ-TCD powder are in FIGS. 9A and B, respectively). The spectrum of α-TCD powder in 2A and of β- and γ-TCD powder in 9A and B are very similar to one another which is as expected because these three compounds differ only in the number of glucose moieties making up the ring. The dominant absorption bands of these spectra are those due to C═O stretching vibration at ˜1746 cm⁻¹; medium and weak hands at ˜1372 cm⁻¹ and 1434 cm⁻¹ can be attributed to the symmetric and asymmetric deformation of CH₃ group of acetates, C—O asymmetric stretching vibration of acetates at ˜1216 cm⁻¹ and the asymmetric stretching vibration of the O—CH₂—C groups for acetates.^{21,47,48}

Also included is the FT-IR spectrum of the 50:50 CS:α-TCD composite film. In addition to bands due to CS, the composite material also exhibits, as expected, all bands which are due to the α-TCD as described above.

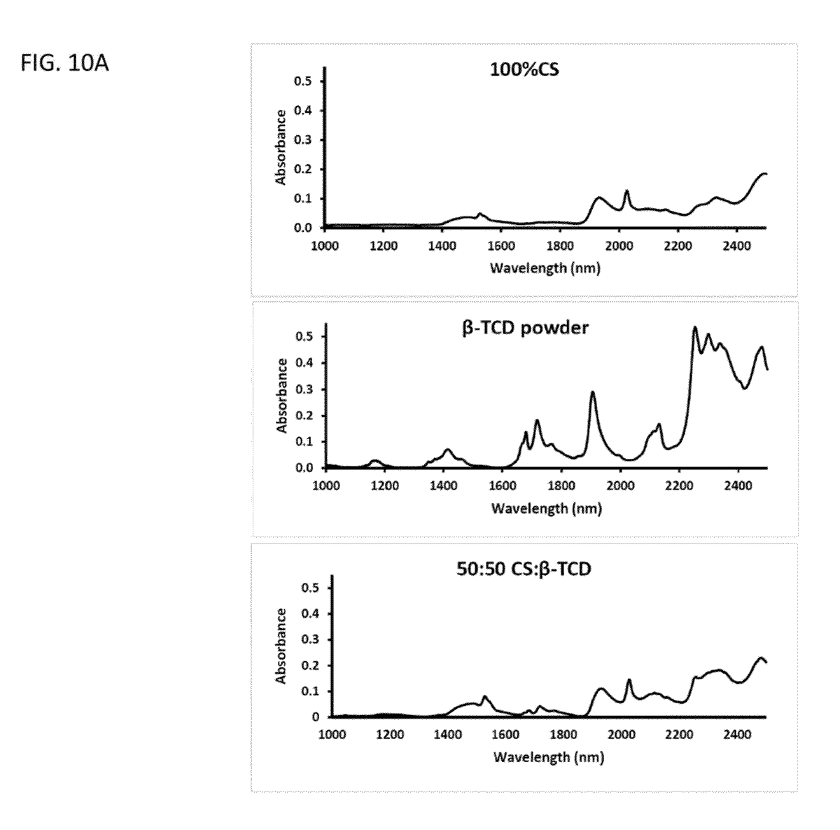

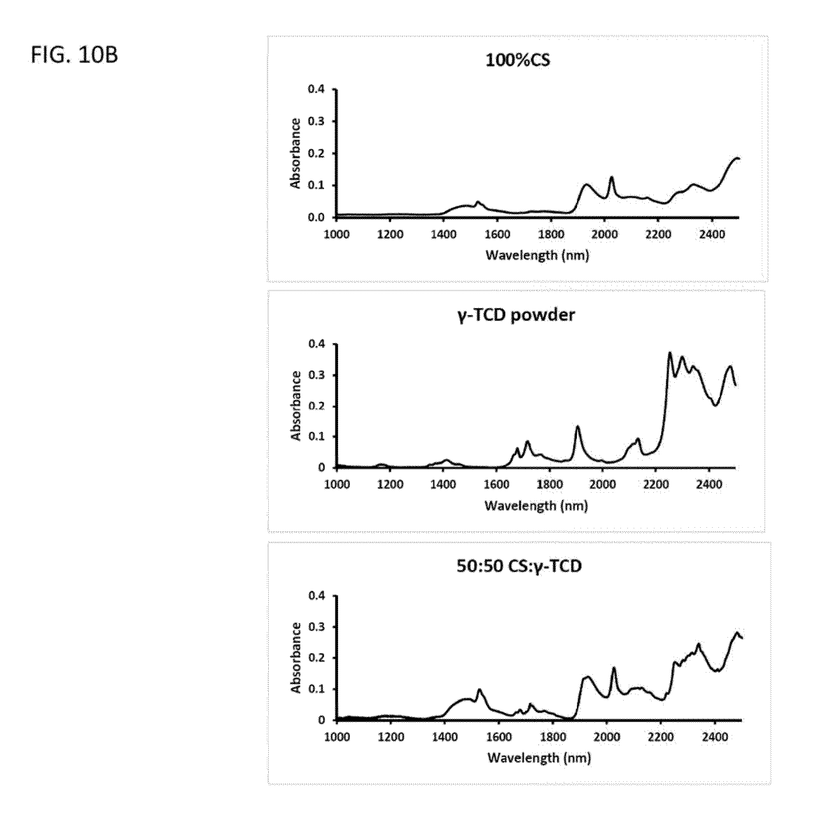

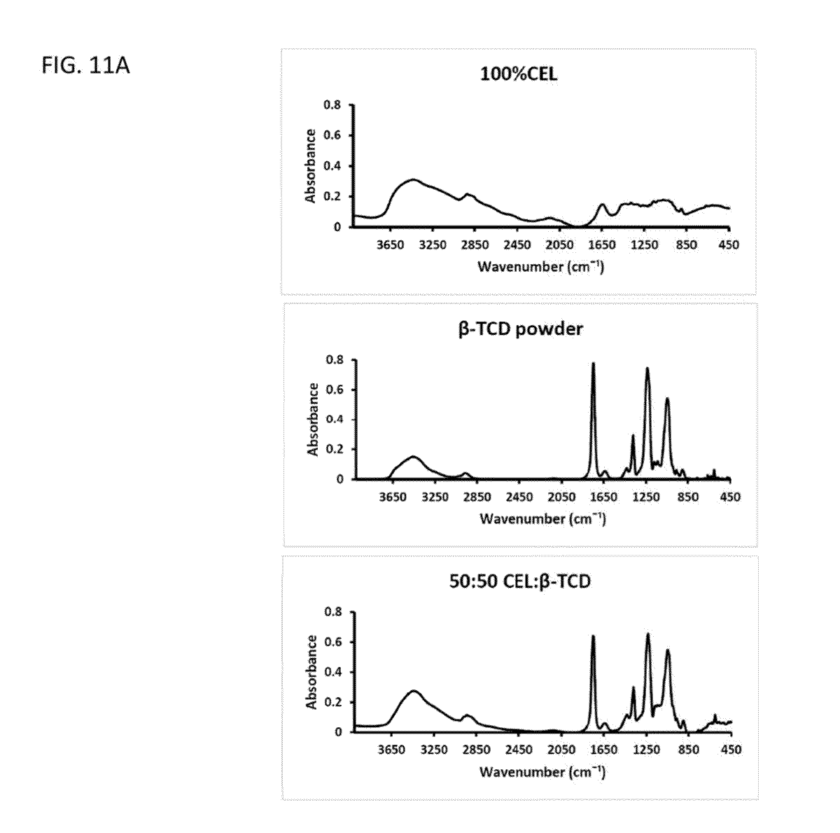

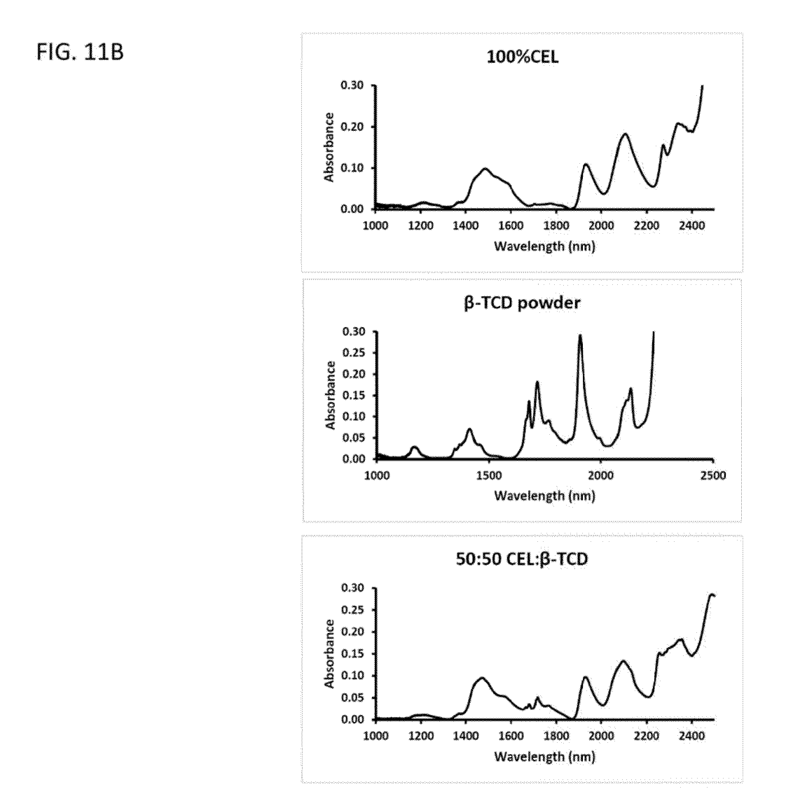

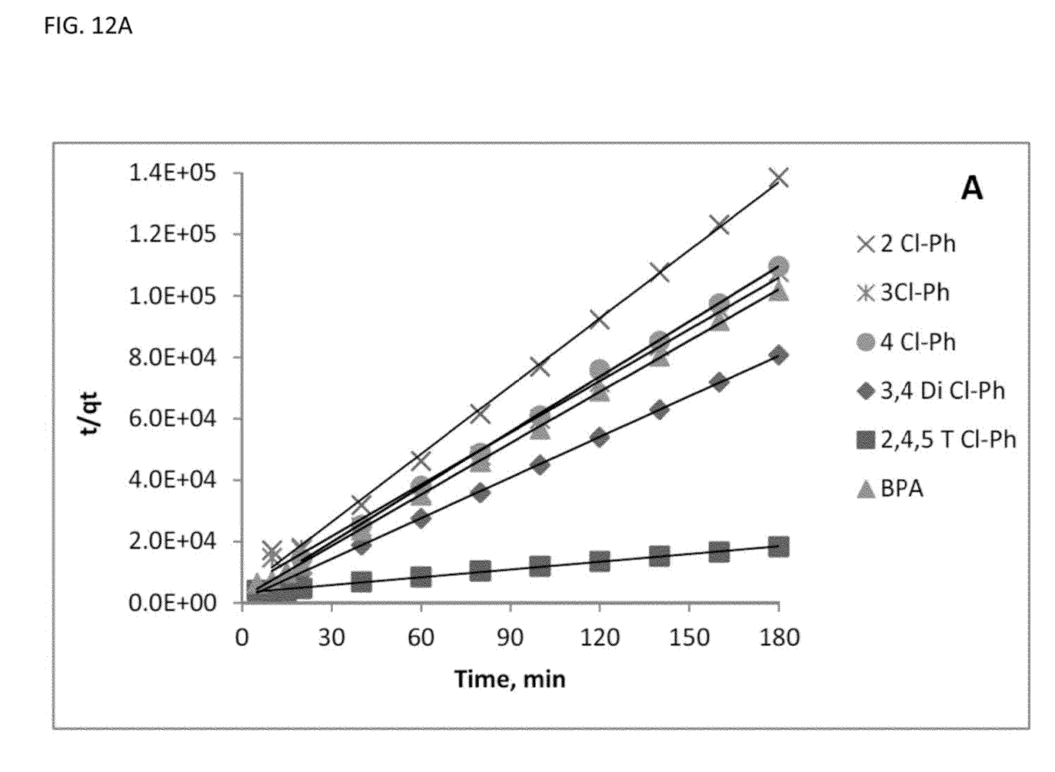

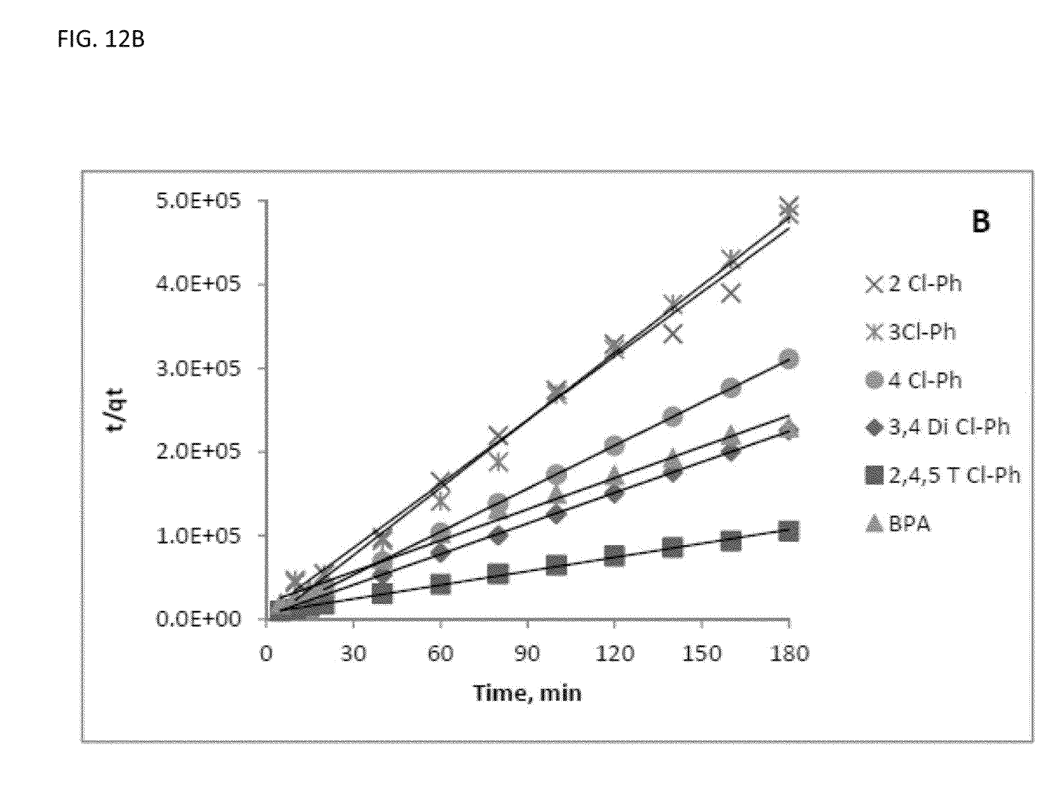

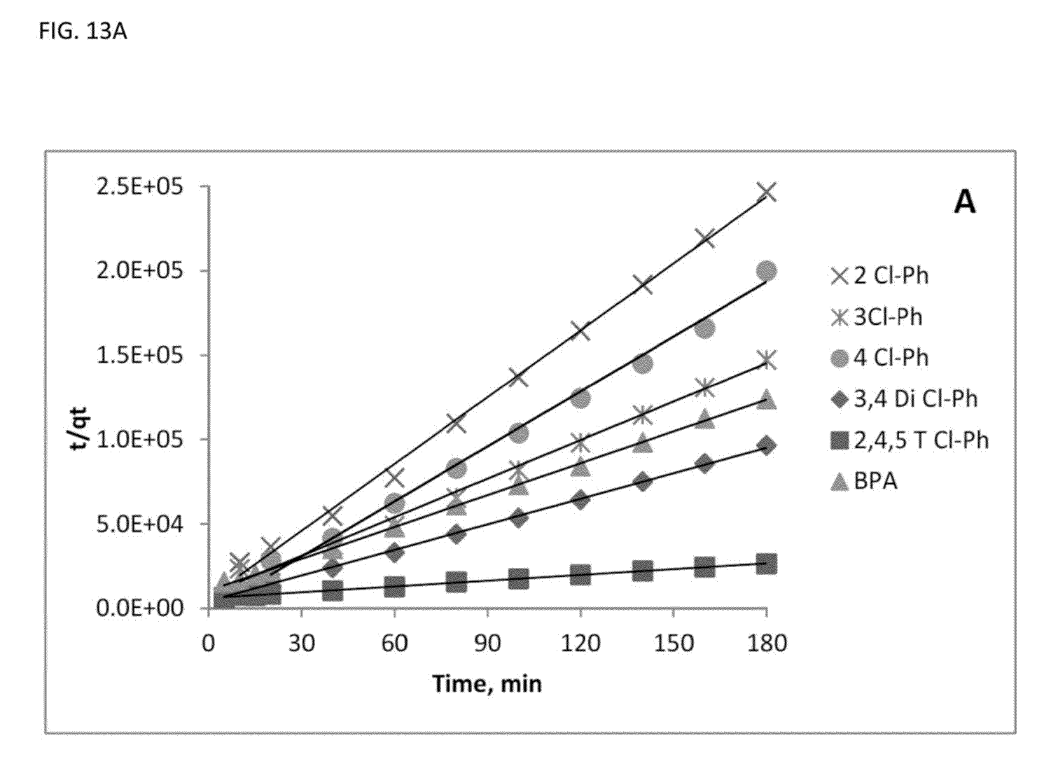

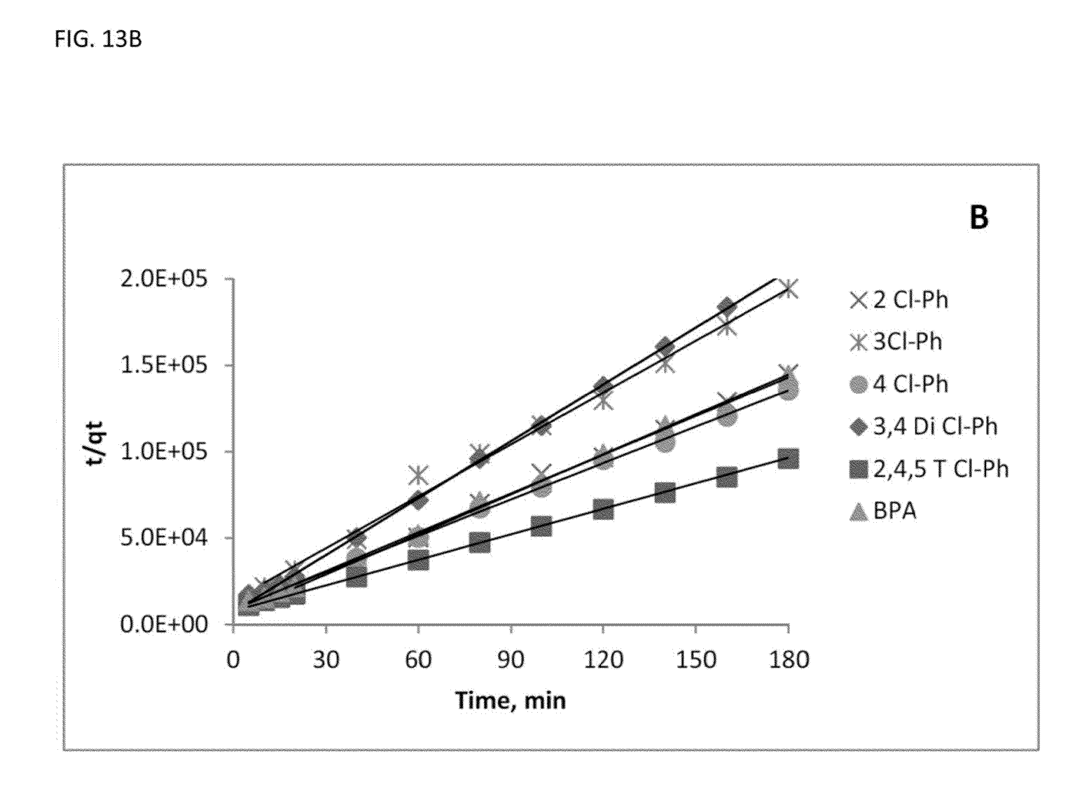

Results from NIR measurements further confirm the successful incorporation of the TCDs into CS (FIGS. 2B and 10) and CEL (FIG. 11). The 100% CS film exhibits NIR absorption bands around 1492 nm, 1938 nm and 2104 nm (FIG. 2B) which can be assigned to the overtone and combination transitions of the —OH group.^{21,28,46,48} In addition, CS also exhibits bands ˜1548 nm and 2028 nm, which is due to the —NH groups.⁴⁹

Similar to FT-IR, the NIR spectra of α-, β- and γ-TCD are also very similar. The major bands for these are around 1415 nm (first overtone of methyl —CH group), 1680 nm and 1720 nm (first overtone of —CH group), 1908 nm and 2135 nm (—C═O, acetyl group).⁵⁰ As shown in FIG. 2B (and FIGS. 10A and B), the NIR spectra of [CS+α-TCD], [CS+β-TCD] and [CS+γ-TCD] composite materials contain bands due to both CS and TCDs.

Similarly, FT-IR and NIR results also confirm that α-TCD, β-TCD and γ-TCD were successfully incorporated into CEL. For clarity, FT-IR and NIR spectra of only β-TCD powder, 50:50 CEL:β-TCD together with 100% CEL film are shown in FIGS. 11A and B, respectively. 100% CEL film (FIG. 11A) exhibits three pronounced bands at around 3400 cm⁻¹, 2850-2900 cm⁻¹ and 890-1150 cm⁻¹. These bands can be tentatively assigned to stretching vibrations of O—H, C—H and —O-group, respectively.⁴⁴ Similar to CS composite materials, FT-IR and NIR spectra of [CEL+β-TCD] composite material (as well as [CEL′α-TCD] and [CEL+γ-TCD] composites, spectra not shown) also exhibit bands due to both TCDs and CEL.

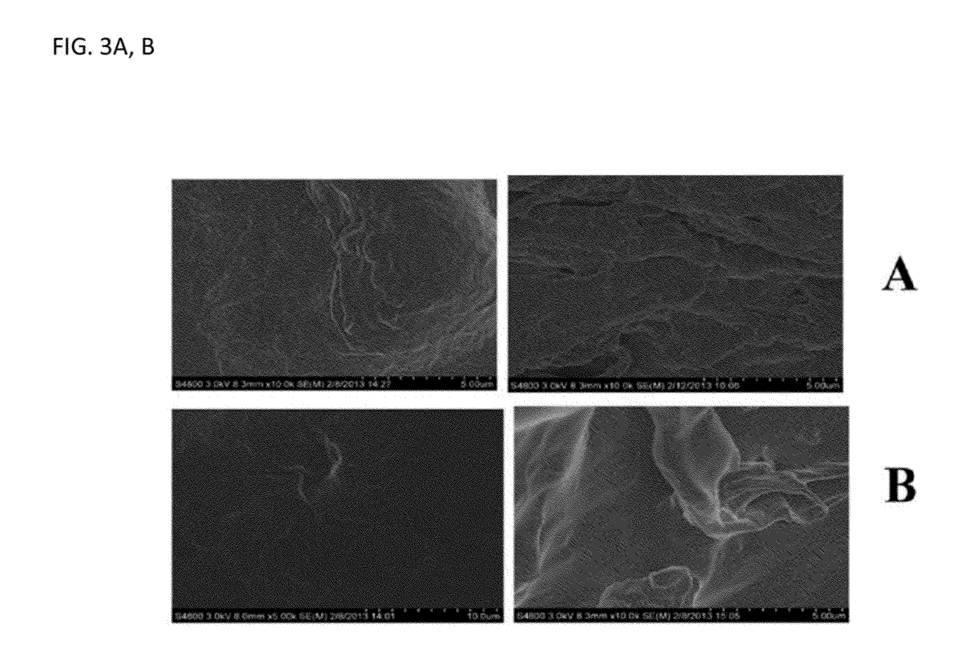

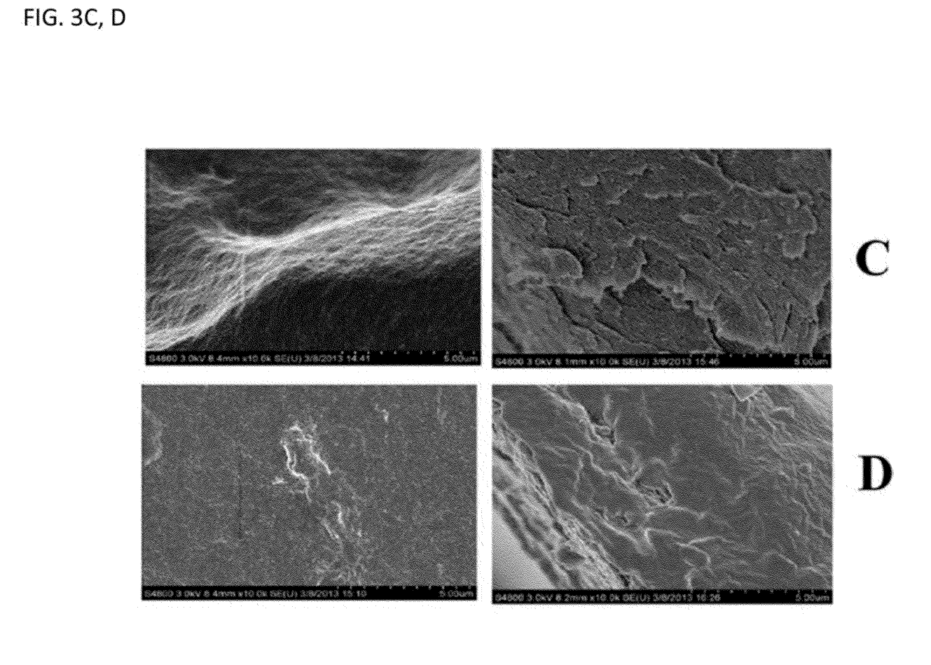

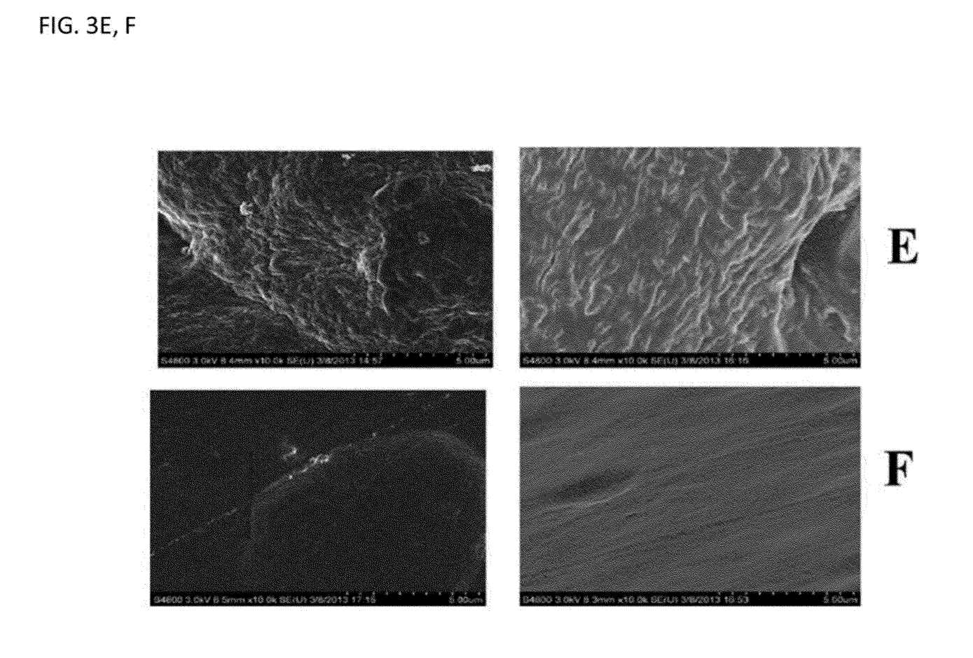

Analysis of the composite: materials by SEM reveals some interesting features about the microstructure of the materials. Shown in FIG. 3 are surface (images on left column) and cross section images (images on right column) of regenerated one component 100% CEL and 100% CS film (first and second row) and 50:50 [CEL+γ-TCD], [CEL+β-TCD], [CS+γ-TCD]. and [CS+β-TCD] (row 3-6). As expected, both surface and cross section images clearly indicate that one-component CEL and CS are homogeneous. Chemically, the only difference between CS and CEL is amino in the former. However, their structures, as recorded by the SEM are substantially different. Specifically, while CS exhibits a rather smooth structure, CEL seems to arrange itself into fibrous structure with fibers having diameter of about ˜0.5-1.0 micron. Interestingly, the structure of a 50:50 composite between CS and γ-TCD (images on row 5) seems to be very much different from that of the 50:50 [CS+β-TCD] (images on row 6). SEM images of the latter seem to indicate that it has rather smooth structure which is different from the rather fibrous structure of the 50:50 [CS+γ-TCD] composite. Similarly, the microstructure of the 50:50 [CEL+γ-TCD] (row 3) is also different from that of [CEL+β-TCD]. It is known that β-CD, being relatively small, has a rather rigid structure whereas the large γ-CD has a more flexible structure. Also γ-CD is very soluble in water (23.2 g/100 mL of water) whereas β-CD can hardly dissolve in water (1.85 g/100 mL). It is possible that because of these difference, when β-TCD forms a composite with either CS or CEL, it will adopt a microstructure which is much different from that of a composite between γ-TCD with either CEL or CS.

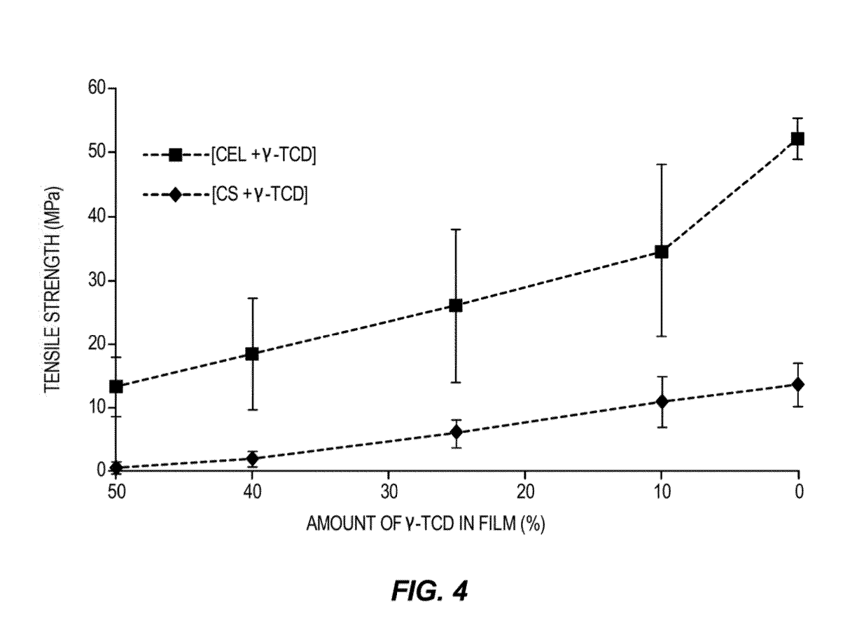

As described above, mechanical and rheological strength of CDs is so poor that practically they cannot: be fabricated into films for practical applications. Measurements were made to determine tensile strength of [CEL+TCDs] and [CS+TCDs] composite films with different CEL and CS concentrations in order to determine if adding CEL or CS would provide the composite material adequate mechanical strength for practical applications Results obtained and shown in FIG. 4 clearly indicate that adding either CEL or CS into the composite materials substantially increase their tensile strength. For example, up to 2× (or 6×) increase in tensile strength can be achieved by increasing concentration of CEL in [CEL+γ-TCD] composite (or CS in [CS+γTCD]) from 50% to 75%. Also, the tensile strength of the [CEL+γ-TCDs] composite is relatively higher than the corresponding [CS+γ-TCDs] composite. This is hardly surprising considering the fact that the mechanical and rheological strength of CEL is relatively higher than that of CS. It is thus, evidently clear that the [CEL+TCD] and [CS+TCD] composite materials have overcome the major hurdle currently imposed on utilization of the materials, namely they have the required mechanical strength for practical applications.

Taken together, XRD, FT-IR, NIR and SEM results presented clearly indicate that novel all polysaccharide composite materials containing CEL, CS and α-TCD, β-TCD and γ-TCD were successfully synthesized by use of [BMIm⁺Cl⁻], an ionic liquid, as the sole solvent. Since majority (at least 88%) of [BMIm⁺Cl⁻] used was recovered for reuse, the method recyclable. As anticipated, adding CEL (or CS) into the composites substantially increases mechanical strength of the composites. It is expected that the composites may also retain properties of CS and TCDs namely, they would be good adsorbent for pollutants (from CS) and selectively form inclusion complexes with substrates of different sizes and shapes (from TCDs). Initial evaluation of their ability to selectively adsorb various endocrine disruptors including polychlrophenols and bisphenol A is described in following section.

### Adsorption of Endocrine Disruptors (2-, 3-, and 4-chlorophenol 3,4-dichlorophenol, 2,4,5-trichlorophenol and bisphenol A)

Adsorption Kinetics. Experiments were designed to determine: (1) if CEL, CS, [CEL+TCD] and [CS+TCD] composite materials can adsorb chlorophenols and bisphenol A; (2) if they can, rate constants, adsorbed amounts at equilibrium (qₑ) and mechanism of adsorption processes; (3) composite material which gives highest adsorption; and (4) if TCDs can provide any selectivity on adsorption of analytes with different sizes and shapes. These were accomplished by initially fitting kinetic data to both pseudo-first order and pseudo-second order models. Appropriate reaction order for the adsorption processes was determined based on the correlation coefficients (R²) and the Model Selection Criteria (MSC) values. Rate constants and qₑ values were then obtained from the kinetic results.^{51,52} Subsequent fitting of data to intra-particle diffusion model together with results of adsorption isotherms measurements yielded additional insight into adsorption process.

The pseudo first order and pseudo second order kinetic models were used to obtain the rate constants and equilibrium adsorption capacity of 100% CEL 100% CS 50:50 CS:β-TCD and 50:50 CEL:β-TCD composite materials for different analytes including chlorophenols and bisphenol A. Results obtained by pseudo-1^{st} order and pseudo-2^{nd} order fitting of adsorption of all analytes by 100% CEL, 100% CS, 50:50 CS:β-TCD and 50:50 CEL:β-TCD are listed in Tables 3-6. In all cases, the R² and the MSC values are higher for the pseudo-2nd order kinetic model than those corresponding for the pseudo first order kinetic model. In addition, the theoretical and experimental equilibrium adsorption capacities, qₑ, obtained for the pseudo-1St order kinetic model varied widely for the different analytes. The results seem to suggest that the adsorption of various chlorophenols and BPA onto 100% CEL, 100% CS, 50:50 CS:β-TCD and 50:50 CEL:β-TCD composite materials is best described by the pseudo-2nd order kinetic model. Good correlation of the system provided by the pseudo-2nd order kinetic model suggest that chemical sorption involving valence forces through sharing or exchange of electrons between adsorbent and analyte might be significant.⁵²

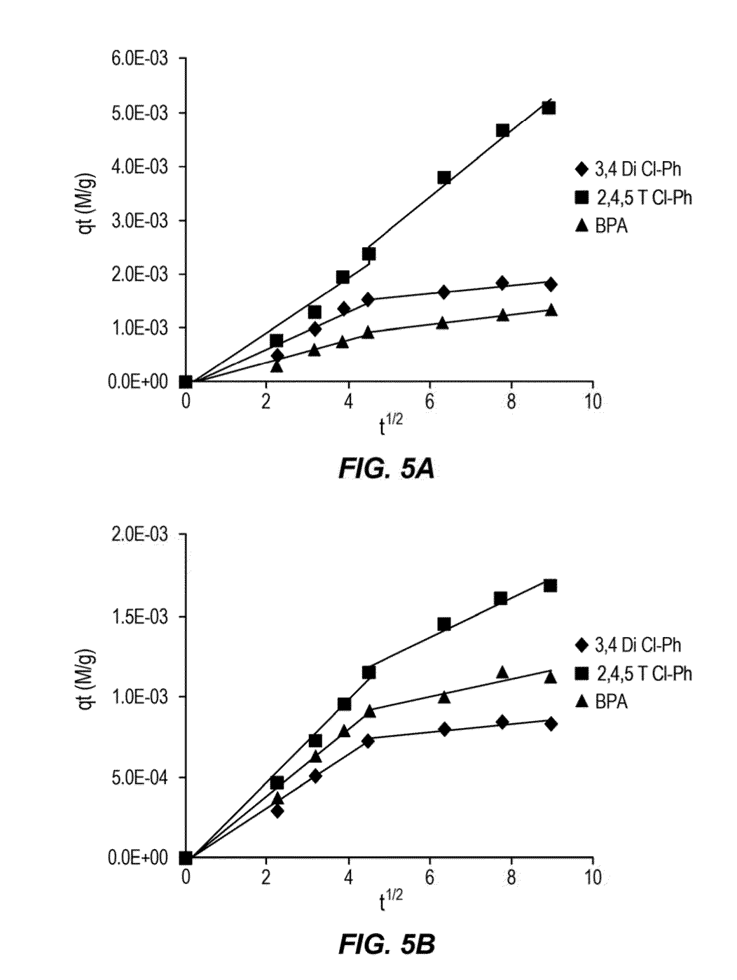

Additional information on mechanism of adsorption can be gained by analyzing data using the intra-particle diffusion model described in the Experimental Section above. Shown in FIGS. 5A and B are representative intra-particle pore diffusion plots (qₜ versus t^{1/2}) for three of the analytes studied, 3,4-Di-Cl-Ph, 2,4,5-Tri-Cl-Ph and BPA adsorbed on 50:50 CEL: β-TCD and 50:50 CS: β-TCD composites. As illustrated, plots of qₜ versus t^{1/2} are not linear but rather non-linear which can be fitted to two linear segments for all analytes on both composites with the exception that data for 2,4,5-Tri-Cl-Ph on 50:50 CS: β-TCD may possibly be fitted to a linear regression with R²=0.9819. According to this model, the 1st sharper linear region can be assigned to the instantaneous adsorption or external surface adsorption, while the second region may be due to gradual adsorption stage where intra-particle diffusion is the rate limiting.^{51,53} These results seem to imply that the intra-particle diffusion is not the sole rate controlling step but other mechanisms may also contribute to the adsorption process.

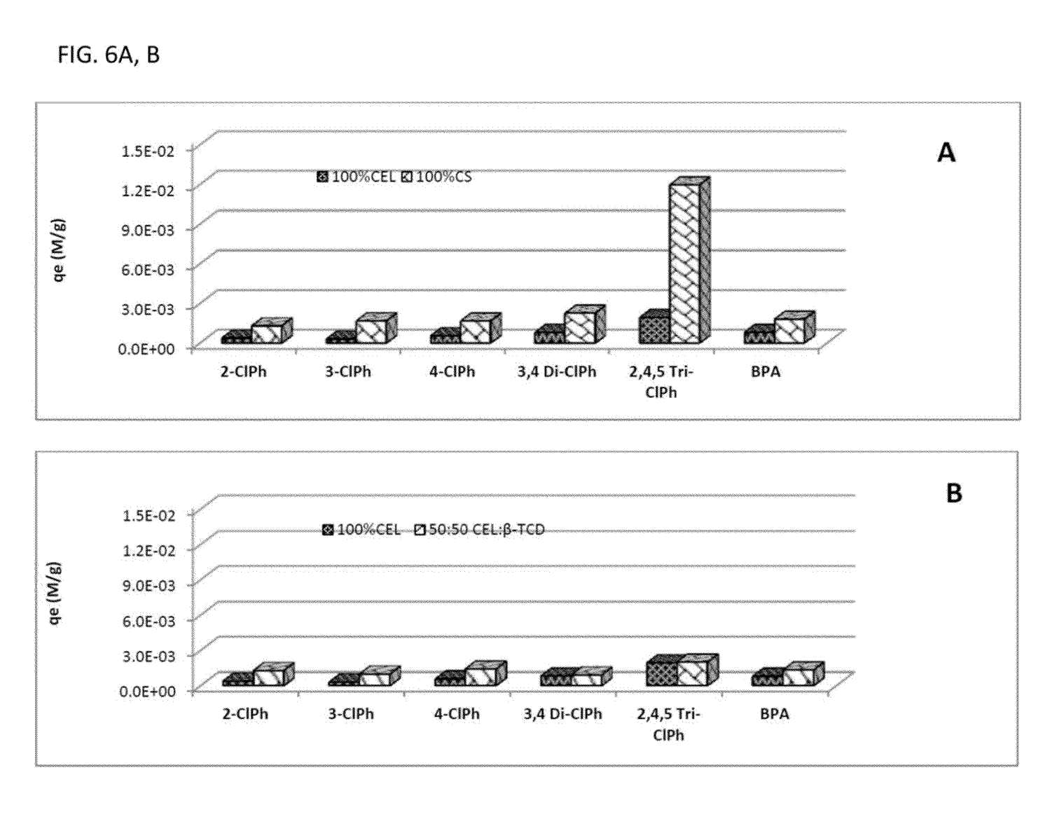

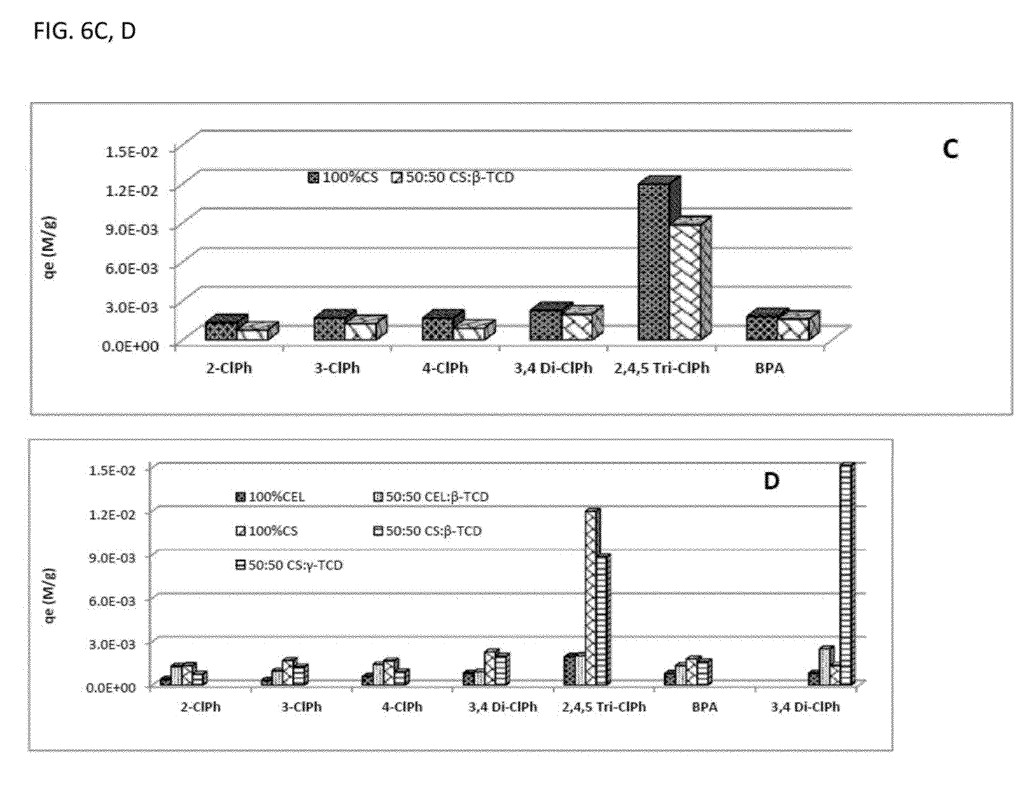

Results obtained from the pseudo-2nd order kinetics in terms of equilibrium sorption capacity (qₑ) and rate constant (k₂) were then used to evaluate sorption performance of the composite materials. Table 1 lists qₑ and k₂ values for 5 different chlorophenols and BPA on 100% CEL, 50:50 CEL:β-TCD, 100% CS and 50:50 CS:β-TCD, respectively. For clarity of presentation and discussion, data from the tables were used to construct three plots for three pairs of composites: 100% CEL and 100% CS (FIG. 6A), 100% CEL and 50:50 CEL:β-TCD (FIG. 6B) and 100% CS and 50:50 CS:β-TCD (FIG. 6C). FIG. 6D plots results obtained for all analytes by all composite materials.

It is evident from FIG. 6A that, for all analytes, equilibrium sorption capacities for 100% CS material are much higher than those corresponding for 100% CEL material e.g., for 2,4,5-Tri-Cl-Ph, the 100% CS material exhibits up to 6× more equilibrium sorption capacity than the 100% CEL material. Even for BPA, where the difference between CEL and CS materials are smallest, the CS material still has a qₑ value twice as much as that of the CEL material. This is as expected, because CEL is known to be inert whereas CS is reported to be an effective adsorbent for various pollutants.

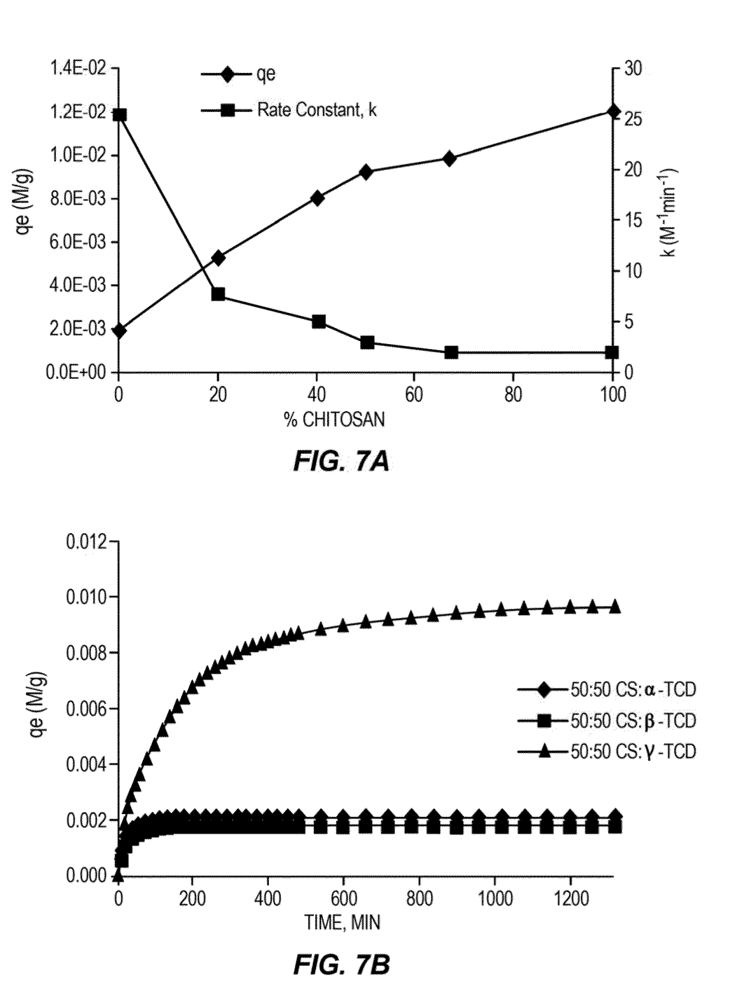

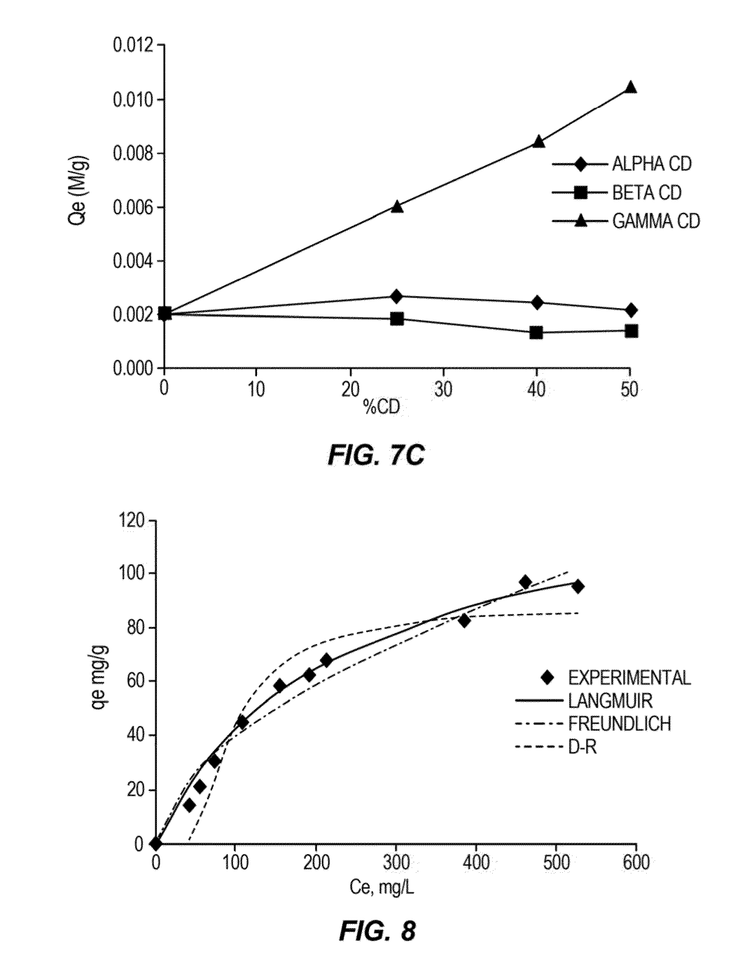

Additional experiments were then performed to further confirm these results. Specifically, six different [CEL+CS] composite materials with different CEL and CS compositions were synthesized, and their adsorption of 2,4,5 trichlorophenol was measured. Results obtained, in terms if qₑ and k₂ values, plotted as a function of CS concentration in the composites are shown in FIG. 7A. It is evident that adding CS into CEL resulted in improved uptake of 2,4,5 trichlorophenol. For example, qₗₑ value was increased by 5 folds when 50% of CS was added to CEL, and the equilibrium sorption capacity seems to be proportional to concentration of CS in the composite. These results clearly indicate that CS is responsible for adsorption of the endocrine disruptors, and that sorption capacity of a composite toward endocrine disruptors can be set at any value by judiciously adjusting concentration of CS in the composite.

When added to CEL. β-TCD: seems to exert much different effect on equilibrium sorption capacity than that of CS. As illustrated in FIG. 6B, substantial enhancement in qₑ values was observed when 50% of β-TCD was added to CEL, but the enhancement was riot observed for all analyte (as were seen for CS) but only for four analytes; i.e., about 3-fold enhancement for 2- and 3-chlorophenol and about 2× for 4-chlorophenol and bisphenol-A. Within experimental error, no observable enhancement was observed for 3,4-dichlorophenol and 2,4,5-trichlorophenol when 50% of β-TCD was added to CEL. A variety of reasons might account for lack of enhancement for 3,4-dichloro and 2,4,5-trichlorophenol but the most likely one is probably due to the bulky dichloro- and trichloro groups on these compounds which sterically hinders their ability to form inclusion complexes with β-TCD.

To further investigate difference effect of CS and β-TCD on adsorption process, adsorption results by 100% CS and 50:50 CS: β-TCD for all analytes were plotted in FIG. 6C. Compared to β-TCD, CS has relatively higher qₑ values for all analytes including 3,4-dichloro- and 2,4,5-trichlorophenol. The latter two compounds, as described above, may not be able to be included in the cavity of β-TCD because of their bulky groups. The results seem to indicate that CS may adsorb the analytes by mechanism which is different from that of the β-TCD, namely surface adsorption appears to be the main and only adsorption mechanism for CS whereas the inclusion complex formation seems to be the main adsorption process for β-TCD with surface adsorption being the secondary mechanism. It is expected that While efficiency for surface adsorption by CS is relatively higher than that of inclusion complex formation, it may not provide any selectivity due to size and shape of host as well as guest compounds. To investigate this possibility, adsorption of 3,4-dichlorophenol by 50:50 CS:γ-TCD as well as by 100% CEL, 100% CS, 50:50 CEL: β-TCD and 50:50 CS: β-TCD were measured and results are presented as the last group on the far right of FIG. 6D. As expected, results obtained further confirm the proposed mechanism. Specifically, 3,4-dichlorophenol, as described in previous section, because of its bulky dichloro group, cannot form inclusion complexes with β-TCD. Therefore, it was adsorbed by CS as well as by β-TCD mainly through surface adsorption. Conversely, γ-TCD with its cavity about 58% larger than that of β-TCD, can well accommodate 3,4-dichlorophenol to its cavity through inclusion complex formation which leads to substantial enhancement in adsorption capacity for 50:50 CS:γ-TCD as compared to other composites.

Additional evidence to confirm inclusion complex formation and size selectivity provided by TCD is shown in FIG. 7B which plots the sorption profiles for 3,4-dichlorophenol by 50:50 CS:α-TCD, 50:50 CS:β-TCD and 50:50 CS:γ-TCD. As expected, because the cavities of α-TCD and β-TCD are too small to accommodate 3,4-dichlorophenol, the latter can only be adsorbed onto 50:50 CS:α-TCD and 50:50 CS:β-TCD by surface adsorption which led to low and similar adsorption curve for both composite materials. Conversely, 50:50 CS:γ-TCD with its larger γ-TCD, can readily form inclusion complexes with 3,4-dichlorophenol, and as a consequence, can adsorb much more analyte, i.e. substantially higher sorption profile.

Plot of equilibrium sorption capacity (qₑ) for 3,4-dichlorophenol by CS+α-TCD, CS-β-TCD and CS+γ-TCD as a function of α-, β- and γ-TCD concentration in the composites are shown in FIG. 7C. Again, since 50:50 CS:α-TCD and 50:50 CS:β-TCD cannot form inclusion complex with 3,4-dichlorophenol, their qₑ values remain the same regardless of the concentration of α- and β-TCD in the composite materials. Not only that qₑ profile of CS+γ-TCD material is different and much higher than those for CS+α-TCD and CS+β-TCD but also qₑ value is proportional to the concentration of γ-TCD in the composite material. For example, adding 50% γ-TCD to CS material led up to 5 folds increase in qₑ value. This is probably due to the fact that because γ-TCD can readily form inclusion complexes with 3,4 dichlorophenol, increasing concentration of γ-TCD in the [CS+γ-TCD] material resulted in higher concentration of inclusion complexes, and hence higher qₑ value.

Adsorption isotherms. To gain insight into adsorption process, investigation was then earned out to determine adsorption isotherm for adsorption of 3,4-dichlorophenol by 100% CS and 50:50 CS: γ-TCD. These two composites were selected because kinetic results presented above indicate that they adsorb 3,4-dichlorophenol by two distinct different mechanisms: surface adsorption and inclusion complex formation. Experimental results were fitted to three different models. Langmuir isotherm⁵⁴, Freundlich isotherm⁵⁵ and the Dubinin-Radushkevich (D-R) isotherm,^{56,57} described above in the Experimental Section. Fitting of experimental values to these three models is shown in FIG. 8. The parameters obtained from fits to these models are listed in Table 2. As listed, experimental values fit relatively well to theoretical models. For example, R² values for fit of 100% CS and 50:50 CS:γ-TCD composites to the Langmuir, the Freundlich and the D-R model were found to be 0.977 and 0.984, 0.970 and 0.949, and 0.972 and 0.912, respectively. Relatively good agreement was also found for the saturation adsorption capacity q values obtained with the Langmuir model and the D-R model: 137.6 mg/g and 102.6 mg/g by 50:50 CS:γ-TCD, and 63.2 mg/g and 26.7 mg/g by 100% CS. The good fit between the Langmuir isotherm model and the experimental data suggests that the sorption is monolayer; sorption of each molecule has equal activation energy and that the analyte-analyte interaction is negligible.⁵⁸

Additional information on the adsorption process can be obtained from the Freundlich isotherm model, particularly from the constant n in Eq SI-8 because it is blown to be a measure of the favorability of the sorption process.⁵⁸ Because n was found to be 1.0 and 1.4 for 100% CS and 50:50 CS:γ-TCD, respectively, the adsorption of 3,4 dichlorophenol by the latter seems to be more favorable than that of the former.

From the fitting to Dubinin-Radushkevich isotherm model, the mean free energy E values of the sorption process per mole of 3,4 di Cl-Ph were found to be 2.5 KJ/mol and 13.9 KJ/mol for 100% CS and 50:50 CS:γ-TCD, respectively. According to this theory, the sorption of 3,4 di Cl-Ph onto 50:50 CS:γ-TCD composite film is chemisorption and is much stronger than onto 100% CS which is more by physisorpiton. This finding is as expected because as described above, 50:50 CS:γ-TCD composite material can readily form inclusion complexes with 3,4-dichlorophenol, and adsorption by inclusion complex formation is relatively stronger and is chemisorb by nature compared to 100% CS which can adsorb the analyte only by surface adsorption.

Taken together, adsorption isotherm results fully support kinetic results. Specifically, both results clearly indicate that 50:50 CS:γ-TCD with its ability to form inclusion complexes with 3,4-dichlorophenol, can strongly and effectively adsorb much more analyte compared to 100% CS which can only adsorb by surface adsorption which is relatively weaker and less effective.

### Conclusions

In summary, we have successfully developed a novel, simple and one step method to synthesize novel, high performance supramolecular polysaccharide composite materials from CEL, CS and α-, β- and γ-TCD. [BMIm⁺Cl⁻], an ionic liquid (IL), was used as a sole solvent for dissolution and preparation of the composites. Since majority (more than 88%) of [BMIm⁺Cl⁻] used was recovered for reuse, the method is recyclable. The [CEL/CS+TCDs] composites obtained retain properties of their components, namely superior mechanical strength (from CEL), excellent adsorption capability for pollutants (from CS) and ability to selectively forum inclusion complexes with substrates with appropriate sizes and shapes (from γ-TCDs). Specifically, both CS- and TCD-based composite materials can effectively adsorb pollutants such as endocrine disruptors, e.g., chlorophenols and bisphenol A. While CS-based composites can effectively adsorb the pollutants, its adsorption is independent on the size and structure of the analytes. Conversely, the adsorption by TCD-based composites exhibits strong, dependency on size and structure of the analytes. For example, while all three TCD-based composites (i.e., α-, β- and γ-TCD) can effectively adsorb 2-, 3- and 4-chlorophenol, only γ-TCD-based composite can adsorb analytes with bulky groups including 3,4-dichloro- and 2,4,5-trichlorophenol. Furthermore, equilibrium sorption capacities for the analytes with bulky groups by γ-TCD-based composite are much higher than those by CS-based composites. These results together with results from adsorption kinetics and adsorption isotherm clearly indicate that γ-TCD-based composite with its relatively larger cavity size can readily form inclusion complexes with analytes with bulky groups, and through inclusion complex formation, it can strongly adsorb much more analytes and with size/structure selective compared to CS-based composites which can adsorb the analyte only by surface adsorption. For example, up to 138 mg of 3,4-dichlorophenol can be adsorbed by 1 g of 50:50 CS:γ-TCD composite material compared to only 63 mg of 3,4-dichlorophenol per 1 g of 100% CS material. Preliminary result presented in this study are very encouraging and dearly indicate that higher adsorption efficiency can be obtained by judiciously modifying experimental conditions (e.g., replacing film of composite materials with microparticles to increase surface area). Furthermore, since all composite materials used in this study (CEL, CS, CEL+TCD, CS+TCD) are chiral because of their glucose and glucosamine units in CEL, CS and TCD, it is expected that they may exhibit some stereospecificity in the adsorption of chiral analytes. These possibilities are the subject of our current intense study.

### REFERENCES

(1) Katsuhiko, A.; Kunitake, T. Supramolecular Chemistry-Fundamentals and Applications; Springer: Berlin, Heidelberg, 2006.

(2) Rebek, Jr., J. Introduction to the molecular recognition and self-assembly special feature. Proc. Nat. Acad. Sci. 2009, 106, 10423-10424.

(3) Rebek, Jr., J. Molecular Behavior in Small Spaces. Acct. Chem. Res. 2009, 42, 1660-1668.

(4) Hinze, W. L.; Armstrong, D. W, Organized surfactant assemblies in separation science ACS Symposium Ser. 1987, 342, 2-82.

(5) Fakayode, S. O.; Brady, P. N.; Pollard, D. A.; Mohammed, A. K.; Warner, I. M. Multicomponent analyses of chiral samples by use of regression analysis of UV-visible spectra of cyclodextrin guest-host complexes. Anal. Bioanal. Chem. 2009, 394, 1645-1653.

(6) Fakayode, S. O.; Lowry, M.; Fletcher, K. A,; Huang, X.; Powe, A. M.; Warner, I. M. Cyclodextrins host-guest chemistry in analytical and environmental chemistry. Cur. Anal. Chem. 2007, 3, 171-181.

(7) Kitaoka, M.; Hayashi, K. Adsorption of bisphenol A by cross-linked β-cyclodextrin polymer. J. Incl. Phe. Macro. Chem. 2002, 44 429-431.

(8) Nishiki, M.; Tojima, T.; Nishi, N.; Sakairi, N. β-Cyclodextrin-linked chitosan beads: preparation and application to removal of bisphenol A from water. Carbohydrate Let. 2000, 4, 61-67.

(9) Augustine, A. V.; Hudson, S. M.; Cuculo, J. A. Cellulose Sources and Exploitation; Ellis Horwood: New York, 1990.

(10) Dawsey, T. R. Cellulosic Polymers, Blends and Composites; Carl Hanser Verlag: New York, 1994.

(11) Masanori, Y.; Shinya, T. DNA-cyclodextrin-inorganic hybrid material for absorbent of various harmful compounds. Mat. Chem. Phys. 2011, 126, 278-283.

(12) Murai, S.; Kinoshita, K.; Ishii, S.; Aoki, N.; Hattori, K. Removal of phenolic compounds from aqueous solution by β-cyclodextrin polymer. Trans. Mat. Res. Soc. Jap 2006, 31, 977-980.

(13) Bordenave, N.; Grelier, S.; Coma, V. Hydrophobization and antimicrobial activity of chitosan and paper-based packaging material. Biomacromolecules 2010, 11, 88-96.

(14) Rabea, E. I.; Badawy, M. E. T.; Stevens, C. V.; Smagghe, G.; Steurbaut, W. Chitosan as antimicrobial agent: Applications and mode of action. Biomacromolecules 2003, 4, 1457-1465.

(15) Burkatovskaya, M.; Tegos. G. P.; Swietlik, E.; Demidova, T. N.; Castano, A. P.; Hamblin, M. R. Use of chitosan bandage to prevent fatal infections developing from highly contaminated wounds in mice. Biomaterials 2006, 27, 4157-4164.

(16) Rossi, S.; Sandri, G.; Ferrari, F.; Benferonic, M. C.; Caramella, C. Buccal delivery of acyclovir from films based on chitosan and polyacrylic acid. Pharm. Dev. Tech. 2003, 8, 199-208.

(17) Jain, D.; Banerjee, R. Comparison of ciprofloxacin hydrochloride-loaded protein, lipid, and chitosan nanoparticles for drug delivery. J. Biomed. Mat. Res. B Appl Biomater. 2008, 86, 105-112.

(18) Ngah, W. S. W. A. N.; Isa. I. M. Comparison study of copper ion adsorption on chitosan, Dowex A-1, and Zerolit 225. J. Appl. Pol. Sci. 1998, 67, 1067-1070.

(19) Cai, J.; Liu, Y.; Zhang, L. Dilute solution properties of cellulose in LiOH/urea aqueous system. J. Pol. Sci. B. Pol. Phys. 2006, 44. 3093-3101.

(20) Fink, H. P.; Weigel, P.; Purz, H. J.; Ganster, J. Structure fomiation of regenerated cellulose materials from NMMO-solutions. Progress Pol. Sci. 2001, 26, 1473-1524.

(21) Tran, C. D.; Duri, S.; Harkins, A. L. Recyclable Synthesis, Characterization, and Antimicrobial Activity of Chitosan-based Polysaccharide Composite Materials J. Biomed. Materials Res. A 2013, 101A (8), 2248-2257.

(22) Tran, C. D.; Lacerda, S. H. P. Determination of Binding Constants of Cyclodextrins in Room Temperature Ionic Liquids by Near-Infrared Spectrometry. Anal. Chem. 2002, 74, 5337-5341.

(23) Han, X.; Armstrong, D. W. Ionic Liquids in Separations. Acc. Chem. Res. 2007, 40, 1079-1086.

(24) Tran, C. D. Ionic Liquids Applications: Pharmaceutical, Therapeutics and Biotechnology. ACS Symposium Series, 2010, 1038, 35-54.

(25) Welton, T. Room-Temperature Ionic Liquids. Solvents for Synthesis and Catalysis. Chem. Rev. 1999. 99, 2071-2083.

(26) Wasserscheid, P.; Welton, T. Ionic Liquids in Synthesis; Wiley-VCH: Weinheim, Germany, 2003.

(27) Baptista, M. S.; Tran, C. D., Gao, G. H. Near Infrared Detection of Flow Injection Analysis by Acousto-Optic Tunable Filter Based Spectrophotometry. Anal. Chem. 1996, 68, 971-976.

(28) Duri, S.; Majoni, S.; Hossenlopp, J. M.; Iran, C. D. Determination of Chemical Homogeneity of Fire Retardant Polymeric Nanocomposite Materials by Near-infrared Multispectral Imaging Microscopy. Anal. Lett. 43, 1780-1789.

(29) Liebert. T. F.; Heinze, T. J.; Edgar, K. J. Cellulose solvents: for analysis, shaping and chemical modification. ACS symposium series 2010, 1033, 299-317.

(30) Baxter, A.; Dillon, M.; Taylor, K.; Roberts, G. Improved method for I.R. determination of the degree of acetylation of chitosan. Int. J. Biol. Macromol. 1992, 14, 166-169.

(31) Domzy, J.; Roberts, G. Evaluation of infrared spectroscopic techniques for analyzing chitosan Makromol. Chem. 1985, 186, 1671-1677.

(32) Berth, G.; Dautzenberg, H. The degree of acetylation of chitosans and its effect on the chain conformation in aqueous solution. Carbohydrate Polymers 2002, 47, 39-51.

(33) Fereira, M. C.; Marvao, M. R.; Duarte, M. L.; Nunes, T. Optimization of the measuring of chitin/chitosan degree of acetylation by FT-IR spectroscopy. Chitin World, [Proceedings from the International Conference on Chitin and Chitosan], 6th, Gdynia, Pol. 1994, 480-488.

(34) Fereira, M. C.; Marvao, M. R.; Duarte M. L.; Nunes, T.; Feio, G. Chitosan degree of acetylation: comparison of two spectroscopic methods (¹³C CP/MAS NMR and dispersive IR). Chitin World, [Proceedings from the International Conference on Chitin and Chitosan], 6th, Gdynia, Pol. 1994. 476-479.

(35) Hirai. A.; Odani, H. Nakajima, A. Determination of degree of deacetylation of chitosan by ¹H NMR spectroscopy. Polymer Bulletin 1991 26, 87-94.

(36) Santiago de Alvarenga, E. Characterization and properties of chitosan. Biotechnology of biopolymers 2011, 91-108.

(37) Percot, A.; Viton, C.; Domand, A. Characterization of shrimp shell deproteinization. Biomacromolecules 2003, 4, 1380-1385.

(38) Li, B.; Zhang, J.; Dai, F.; Xia, W. Purification of chitosan by using sol-gel immobilized pepsin deproteinization. Carbohydrate Polymers 2012, 88, 206-212.

(39) Xu., A. Wang, J.; Wang, H. Effect of anionic structure and lithium salts addition on the dissolution of cellulose in 1-butyl-3-mrthylimidazolium-based ionic liquid solvent systems. Green Chemistry 2010, 12, 268-275.

(40) Xiao, W.; Chen, Q.; Wu, Y.; Wu, T.; Dai, L. Dissolution and blending of chitosan using 1,3-dimethylimidazolium chloride and 1-H-3-methylimidazolium chloride binary ionic liquid solvent. Carbohydrate Polymers 2011, 83. 233-238.

(41) Fendt. S.; Padmanabhan, S.: Blanch, H. W.; Prausnitz, J. M. Viscosities of acetate or chloride based ionic liquids and some of their mixtures with water or other common solvents. J. Chem. Eng. Data. 2011. 56, 31-34.

(42) Swatloski, R. P.; Spear, S. K.; Holbrey, J. D.; Rogers, R. D. Dissolution of cellulose with ionic liquids. J. Am. Chem. Soc. 2002, 124. 4974-4975.

(43) Zhang. H.; Wu, J.; Zhang, J.; He, J. 1-Allyl-3-methyimidazolium chloride room temperature ionic liquid: A new and powerful nonderivatizing solvent, for cellulose. Macromolecules 2005, 38, 8272-8277.

(44) Da Roz, A. L.; Leite, F. L.; Pereiro, L. V.; Nascente, P. A. P; Zucolotto, V.; Oliveira O. N. Jr.; Carvalho, A. J. F. Adsorption of chitosan on spin-coated cellulose films. Carbohydrate Pol. 2010, 80, 65-70.

(45) Dreve, S.; Kacso, I.; Bratu, I. Indrea, E. Chitosan-based delivery systems for diclofenac delivery: Preparation and characterization. J. Phys.: Conference Series 2009 182, 1-4.

(46) Burns, D. A. Ciurczak. E. W. Handbook of Near-Infrared Analysis; Marcell Dekker: New York, 1992.

(47) Socrates, G. Infrared characteristic group frequencies; John-Wiley: New York, 1994.

(48) Bettinetti, G. Sorrenti, M.; Catenacci, L.; Ferrari, F. Rosi, S. Polymorphism, pseudopolymorphism and amorphism of peracetylated α-, β-, and γ-cyclodextrins. J. Parm. Biomed. Anal. 2006, 41, 1205-1211.

(49) Ellis, J. W. Infra-red absorption by the N—H bond II in aryl, alkyl and aryl-alkyl amines. J. Am. Chem. Soc. 1928, 50, 685-695.

(50) Miller, C. E. Chemical Principles of Near-Infrared Technology. In: Williams, P.; Norris, K. (Eds): Near-Infrared technology in the agricultural and food industries. 2^{nd} Edition. American Association of Cereal Chemists, Minnesota, USA, 2001, 19-37.

(51) Weber, J. W.; Morris, J. C. Kinetics of adsorption of carbon from solution. J. Sanit. Eng. Div. Am. Soc. Civ. Eng. 1963, 89, 31-39.

(52) Chakraborty, S.; Chowdhury, S.; Saha, P. D. Adsorption of Crystal Violet from aqueous solution onto NaOH-modified rice husk. Carbohydrate Polymers. 2011, 86, 1533-1541.

(53) Gu, W.; Sun, C.: Liu, Q.: Cul, H. Adsorption of avernectins on activated carbon: Equilibrium, kinetics, and UV-shielding. Trans. Nonferrous Met. Soc. China. 2009, 19, 845-850.

(54) Langmuir, I. The constitution and fundamental properties of solids and liquids. J. Am. Chem. Soc. 1916, 38, 2221-2295.

(55) Freundlich, H. M. F. Over the adsorption in solution. J. Phys. Chem. 1906, 57A, 385-471.

(56) Dubinin, M. M. The potential theory of adsorption of gases and vapours for adsorbents with energetically non-uniform surfaces. Chem. Rev. 1960, 60-266.

(57) Dubinin, M. M.; Radushkevich, L. V. The equation of the characteristic curve of the activated charcoal. Proc. Acad. Sci. USSR Phys. Chem. Sect., 1947, 55, 331-337.

(58) Chowdhury, S.; Chakraborty, S.; Saha, P. Biosorption of Basic Green 4 from aqueous solution by Ananas comosus (pineapple) leaf powder. Colloids and surfaces B: Biointerfaces 2011, 84, 520-527.

(59) Kundu, S.; Gupta, A. K. Arsenic adsorption onto iron oxide-coated cement (IOCC): regression analysis of equilibrium data with several isotherm models and their optimization. Chem. Eng. J. 2006, 122, 96-106.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

TABLE 1
Comparison of pseudo second order kinetic parameters for 100% CEL,
100% CS, 50:50 CEL:β-TCD and 50:50 CS:β-TCD composite material.

| | 100% CEL | | | | 100% CS | | | |
|---|---|---|---|---|---|---|---|---|
| Analyte | qₑ(M/g) | K₂ (M⁻¹ min⁻¹) | R² | MSC | qₑ(M/g) | K₂ (M⁻¹ min⁻¹) | R² | MSC |
| 2-ClPh | 3.93E−04 | 702.3 | 0.9871 | 3.95 | 1.32E−03 | 385.9 | 0.9998 | 8.02 |
| 3-ClPh | 3.20E−04 | 293.8 | 0.9822 | 3.72 | 1.68E−03 | 133.5 | 0.9960 | 5.21 |
| 4-ClPh | 5.81E−04 | 2054.2 | 0.9999 | 9.13 | 1.66E−03 | 214.6 | 0.9996 | 7.52 |
| 3,4 Di-ClPh | 8.19E−04 | 315.6 | 0.9996 | 7.47 | 2.27E−03 | 169.8 | 0.9999 | 8.72 |
| 2,4,5 Tri-ClPh | 1.95E−03 | 25.4 | 0.9967 | 5.32 | 1.20E−02 | 2.1 | 0.9991 | 6.60 |
| BPA | 8.05E−04 | 78.9 | 0.9911 | 4.39 | 1.80E−03 | 168.3 | 0.9995 | 7.24 |
| | 50:50 CEL:β-TCD | | | | 50:50 CS:β-TCD | | | |
| Analyte | qₑ(M/g) | K₂ (M⁻¹ min⁻¹) | R² | MSC | qₑ(M/g) | K₂ (M⁻¹ min⁻¹) | R² | MSC |
| 2-ClPh | 1.30E−03 | 100.2 | 0.9975 | 5.57 | 7.58E−04 | 268.4 | 0.9967 | 5.32 |
| 3-ClPh | 9.87E−04 | 77.9 | 0.9964 | 5.33 | 1.25E−03 | 118.6 | 0.9934 | 4.71 |
| 4-ClPh | 1.41E−03 | 58.0 | 0.9993 | 6.93 | 8.99E−04 | 313.1 | 0.9957 | 5.11 |
| 3,4 Di-ClPh | 9.12E−04 | 160.6 | 0.9992 | 6.84 | 1.99E−03 | 57.1 | 0.9978 | 5.77 |
| 2,4,5 Tri-ClPh | 2.00E−03 | 33.2 | 0.9995 | 7.26 | 8.84E−03 | 2.1 | 0.9996 | 7.41 |
| BPA | 1.34E−03 | 65.5 | 0.9987 | 6.28 | 1.59E−03 | 37.6 | 0.9994 | 7.11 |

TABLE 2
Adsorption Isotherm Parameters for the Adsorption of 3,4-Dichlorophenol onto 100% CS and 50:50 Cs/γ-TCD
Composite Materials
3,4-Dichlorophenol

| | Langmuir Isotherm Parameters | | | Freundlich Isotherm Parameters | | | D-R Isotherm Parameters | | | |
|---|---|---|---|---|---|---|---|---|---|---|
| | q_{m,n}(mg/g) | K_{L}(L/mg) | R² | n | K_{F}(mg/g)(L/mg)l/n | R² | q_{m,n}(mg/g) | β(mmol²J²) | E (kJ/mol) | R² |
| 100% CS | 63.2 | 0.0004 | 0.977 | 1.0 | 0.015185 | 0.970 | 26.7 | 0.0805 | 2.5 | 0.972 |
| 50:50 CS:γ-TCD | 137.6 | 0.0045 | 0.984 | 1.4 | 1.34975 | 0.949 | 102.6 | 0.0026 | 13.9 | 0.912 |

TABLE 3
Kinetic parameters for adsorption of Chlorophenols an BPA onto 100% CS film.

| | qe, expt | Pseudo first-order kinetic model | | | | Pseudo second-order kinetic model | | | |
|---|---|---|---|---|---|---|---|---|---|
| Analyte | (M/g) | qe(M/g) | K₁ ( min⁻¹) | R² | MSC | qe(M/g) | K₂ (M⁻¹ min⁻¹) | R² | MSC |
| 2-ClPh | 1.30E−03 | 1.48E−03 | 0.089 | 0.9865 | 3.305 | 1.32E−03 | 385.9 | 0.9998 | 8.02 |
| 3-ClPh | 1.62E−03 | 3.25E−03 | 0.050 | 0.9745 | 2.669 | 1.68E−03 | 133.5 | 0.9960 | 5.21 |
| 4-ClPh | 1.64E−03 | 6.49E−04 | 0.051 | 0.9849 | 2.861 | 1.66E−03 | 214.6 | 0.9996 | 7.52 |
| 3,4 Di-ClPh | 2.23E−03 | 7.23E−04 | 0.048 | 0.8769 | 0.761 | 2.27E−03 | 169.8 | 0.9999 | 8.72 |
| 2,4,5 Tri-ClPh | 1.05E−02 | 9.90E−03 | 0.016 | 0.9843 | 3.917 | 1.20E−02 | 2.1 | 0.9991 | 6.60 |
| BPA | 1.74E−03 | 5.88E−04 | 0.040 | 0.8947 | 1.680 | 1.80E−03 | 168.3 | 0.9995 | 7.24 |

TABLE 4
Kinetic parameters for adsorption of Chlorophenols an BPA onto 100% CEL film.

| | qe, expt | Pseudo first-order kinetic model | | | | Pseudo second-order kinetic model | | | |
|---|---|---|---|---|---|---|---|---|---|
| Analyte | (M/g) | qe(M/g) | K₁ (min⁻¹) | R² | MSC | qe(M/g) | K₂ (M⁻¹ min⁻¹) | R² | MSC |
| 2-ClPh | 4.11E−04 | 1.45E−04 | 0.029 | 0.6469 | 0.041 | 3.93E−04 | 702.3 | 0.9871 | 3.95 |
| 3-ClPh | 3.19E−04 | 4.95E−04 | 0.044 | 0.9747 | 2.678 | 3.20E−04 | 293.8 | 0.9822 | 3.72 |
| 4-ClPh | 5.79E−04 | 1.69E−04 | 0.055 | 0.9559 | 1.788 | 5.81E−04 | 2054.2 | 0.9999 | 9.13 |
| 3,4 Di-ClPh | 7.98E−04 | 9.44E−04 | 0.142 | 0.9665 | 2.397 | 8.19E−04 | 315.6 | 0.9996 | 7.47 |
| 2,4,5 Tri-ClPh | 1.87E−03 | 1.01E−03 | 0.011 | 0.9714 | 3.287 | 1.95E−03 | 25.4 | 0.9967 | 5.32 |
| BPA | 7.27E−04 | 4.62E−04 | 0.014 | 0.9715 | 3.156 | 8.05E−04 | 78.9 | 0.9911 | 4.39 |

TABLE 5
Kinetic Parameters for Adsorption of Chlorophenols and BPA onto 50:50 CS:β-TCD Composite Material.

| | qe, expt | Pseudo first-order kinetic model | | | | Pseudo second-order kinetic model | | | |
|---|---|---|---|---|---|---|---|---|---|
| Analyte | (M/g) | qe(M/g) | K₁ (min⁻¹) | R² | MSC | qe(M/g) | K₂ (M⁻¹ min⁻¹) | R² | MSC |
| 2-ClPh | 7.30E−04 | 7.76E−04 | 0.058 | 0.9981 | 5.249 | 7.58E−04 | 268.4 | 0.9967 | 5.32 |
| 3-ClPh | 1.22E−03 | 1.96E−03 | 0.041 | 0.9936 | 4.046 | 1.25E−03 | 118.6 | 0.9934 | 4.71 |
| 4-ClPh | 9.64E−04 | 4.79E−03 | 0.119 | 0.9567 | 1.806 | 8.99E−04 | 313.1 | 0.9957 | 5.11 |
| 3,4 Di-ClPh | 1.87E−03 | 1.45E−03 | 0.055 | 0.9636 | 2.647 | 1.99E−03 | 57.1 | 0.9978 | 5.77 |
| 2,4,5 Tri-ClPh | 7.45E−03 | 7.92E−03 | 0.015 | 0.9548 | 2.861 | 8.84E−03 | 2.1 | 0.9996 | 7.41 |
| BPA | 1.42E−03 | 1.12E−03 | 0.028 | 0.9782 | 3.381 | 1.59E−03 | 37.6 | 0.9994 | 7.11 |

TABLE 6
Kinetic parameters for adsorptionof Chlorophenols and BPA onto 50:50 CEL:β-TCD Composite Material.

| | qₑ, expt | Pseudo first-order kinetic model | | | | Pseudo second-order kinetic model | | | |
|---|---|---|---|---|---|---|---|---|---|
| Analyte | (M/g) | qₑ(M/g) | K₁ (min⁻¹) | R² | MSC | qₑ(M/g) | K₂ (M⁻¹ min⁻¹) | R² | MSC |
| 2-ClPh | 1.24E−03 | 8.70E−04 | 0.041 | 0.8960 | 1.597 | 1.30E−03 | 100.2 | 0.9975 | 5.57 |
| 3-ClPh | 9.26E−04 | 5.55E−04 | 0.020 | 0.9410 | 2.259 | 9.87E−04 | 77.9 | 0.9964 | 5.33 |
| 4-ClPh | 1.33E−03 | 1.04E−03 | 0.028 | 0.8161 | 1.122 | 1.41E−03 | 58.0 | 0.9993 | 6.93 |
| 3,4 Di-ClPh | 8.71E−04 | 5.28E−04 | 0.047 | 0.9422 | 2.185 | 9.12E−04 | 160.6 | 0.9992 | 6.84 |
| 2,4,5 Tri-ClPh | 1.92E−03 | 1.31E−03 | 0.021 | 0.9867 | 3.957 | 2.00E−03 | 33.2 | 0.9995 | 7.26 |
| BPA | 1.28E−03 | 7.93E−04 | 0.030 | 0.9291 | 2.147 | 1.34E−03 | 65.5 | 0.9987 | 6.28 |

1. An ionic liquid composition comprising a structural polysaccharide and a macrocyclic compound dissolved in an ionic liquid.

2. The composition of claim 1, wherein the structural polysaccharide is a polymer comprising 6-carbon monosaccharides linked via beta-1,4 linkages.

3. The composition of claim 1, wherein the structural polysaccharide is cellulose.

4. The composition of claim 1, wherein the structural polysaccharide is chitin.

5. The composition of claim 1, wherein the structural polysaccharide is chitosan.

6. The composition of claim 1, wherein the macrocyclic compound is selected from a group consisting of a cyclodextrin, a calixarene, a carcerand, a crown ether, a cyclophane, a cryptand, a cucurbituril, a pillararene, and a spherand.

7. The composition of claim 6, wherein the macrocyclic compound is a cyclodextrin.

8. The composition of claim 7, wherein the cyclodextrin is an α-cyclodextrin, a β-cyclodextrin, or a γ-cyclodextrin.

9. The composition of claim 7, wherein the cyclodextrin is a modified cyclodextrin having one or more substitutions on a hydroxyl group.

10. The composition of claim 9, wherein the substitution is selected from a group consisting of an alkyl group, a hydroxyalkyl group, a sulfoalkyl group, and a sugar group.

11. The composition of claim 9, wherein the modified cyclodextrin is selected from a group consisting of methyl cyclodextrins, hydroxyethyl cylcodextrins, 2-hydroxypropyl cyclodextrins, glucosyl cyclodextrins, a sulfobutyl cyclodextrin, a glucosyl cyclodextrin, and maltosyl cyclodextrin.

12. The composition of claim 1, wherein the ionic liquid is an alkylated imidazolium salt.

13.-15. (canceled)

16. The composition of claim 1, wherein the ionic liquid composition comprises at least 10% w/w of the dissolved structural polysaccharide.

17. A method for preparing a composite material comprising a structural polysaccharide and a macrocyclic compound, the method comprising removing the ionic liquid from the composition of claim 1.

18. (canceled)

19. A composite material prepared by the method of claim 17.

20. A method for removing a contaminant from a stream, the method comprising contacting the stream and the composite material of claim 19.

21. A method for killing or eliminating microbes, the method comprising contacting the microbes with the composite material of claim 19.

22. A method of purifying a compound from a stream, the method comprising contacting the compound with the composite material of claim 19.

23.-25. (canceled)

26. A filter comprising the composite material of claim 19.

27. A bandage comprising the composite material of claim 19.

28.-30. (canceled)
